(19) United States
(12) Patent Application Publication
(10) Pub. No.: US 2008/0160020 A1
(43) Pub. Date: Jul. 3, 2008

(54) Methods For Reducing the Symptoms of Autoimmunity and Inflammation Using Binding Proteins Against Antigens Exposed on Dead or Dying Cells

(76) Inventor: Gregg J. Silverman, Encinitas, CA (US)

Correspondence Address: MANDEL & ADRIANO, 572 EAST GREEN STREET, SUITE 230, PASADENA, CA, 91101, US

(21) Appl. No.: 11/883,873

(22) PCT Filed: Feb. 7, 2006
(86) PCT No.: PCT/US06/04065

§ 371 Date: Aug. 7, 2007

Related U.S. Application Data
(60) Provisional application No. 60/650,458, filed on Feb. 7, 2005.

(51) Int. Cl. A61K 39/395; C07K 16/00; A61P 43/00

(52) U.S. Cl. 424/133.1; 424/139.1; 530/387.3; 530/391.9

(57) ABSTRACT

The present invention relates to a method for inhibiting a disease response in a subject comprising contacting dead or dying cells exposing an antigen selected from the group consisting of a phosphorylcholine (PC) determinant, a phosphatidyl serine (PS) determinant, a MDA determinant, and cardiolipin in the subject, with an antibody or recombinant protein that recognizes and binds the antigen that is exposed on the dead or dying cells, thereby inhibiting the pathologic response in the subject.

Throughout this application various publications are referenced. The disclosures of these publications, in their entireties, are hereby incorporated by reference into this application, in order to more fully describe the state of the art to which the invention pertains.

### FIELD OF THE INVENTION

The invention relates to the use of binding proteins that recognize antigens exposed on dead or dying cells to reduce the symptoms of an immune system disease (e.g., autoimmune diseases and/or inflammatory diseases) associated with impaired removal of dead or dying cells.

### BACKGROUND OF THE INVENTION

Many autoimmune diseases are believed to arise in part because they are associated with impaired removal of dead or dying cells. For example, patients with lupus have been shown to have defects in the capacity to clear apoptotic cells¹. Every day more than 10¹⁰ cells turnover, and if these cell corpses cannot be removed appropriately, then their cell components can induce inflammation within the body. Autoantigens released from these cells can induce autoimmune B cell and T cell responses that can lead to or contribute to the development of pathologic autoimmune disease.

To date, there has not been any reported evidence that a practical therapeutic agent can be developed based on a capacity to enhance in vivo apoptotic clearance. Agents that enhance apoptotic clearance also affect the capacity for interactions between these complexes with apoptotic membrane components on cells and cell breakdown products, and cells as macrophages and dendritic cells, and potentially other cells, and these encounters will diminish, alter, or modulate the intensity or quality of immune and inflammatory responses involving cells of the adaptive and innate immune system.

The in vivo clearance of dead or dying cells is a complicated multi-step process that involves numerous surface ligands, bridging molecules, phagocyte receptors, and signaling transducers (reviewed in²). Because the death process varies in different cells, membrane changes associated with different stages of death, can be identified, as well as distinct pathways for cells in early and late stages of apoptosis^{3;4}. Such apoptosis-associated changes may reflect enzymatic alterations, or oxidant damage from pro-apoptotic events (e.g. mitochondrial events).

As previously shown for phosphatidylserine (PS)⁵, phosphoryl choline (PC) determinants become exposed during early apoptosis, by a process linked to oxidative modification of phospholipids (PL), by enzymatic or non-enzymatic mechanisms⁶. Apoptotic cells are reported to cause different cellular responses than necrotic cells, which may be a result of differences in membrane-associated signals, but recent reports suggest that this could be due to the release from necrotic cells of pro-inflammatory factors, such as HMGB1⁷. In vitro studies have shown that the properties of a macrophage can be altered by interaction with PS⁸, but the practical and therapeutic implications of in vivo interactions remain undefined.

Deficiency states for the early complement factors, C1q, C2, and C4, have long been known to predispose people to lupus-like syndromes, but only recently has this been linked to an impairment in the clearance of dead or dying cells. Importantly, while it was first reported that complement components are spontaneously deposited onto the altered surfaces of apoptotic cells^{9;10}, a more recent report showed that efficient in vitro deposition of C1q actually requires the presence of polyclonal IgM from adult mice¹¹. C1q deposition leads to covalent bonding of C3bi and C4 on immune complex (IC)-associated antigens, which are ligands for CD11b/CD18 (i.e., complement receptor 3, CR3), and to a lesser degree CD11c (i.e., complement receptor 4, CR4). Significantly, there are high levels of CR3 and CR4 on macrophages (Mφ) and dendritic cells (DCs), and interactions mediated through these complement receptors can transduce activating signals that likely modulate cellular activities. There has been no reported evidence on whether all IgM antibodies have these properties, or if such functional activities come from encounters with only a subset of IgM antibodies with certain defined binding specificities.

Supporting the hypothesis that circulating IgM can affect overall immune homeostasis, mice deficient in circulating IgM are reported to develop lupus-like autoimmune disease^{12;13}. Moreover, in vitro studies have shown that incubation with human polyclonal IgM antibodies can flag cellular debris for clearance, by facilitating the deposition of complement components¹⁴, which is consistent with past reports that polyclonal IgM can aid the clearance of senescent red cells¹⁵. It has been reported that some antibodies within polyclonal human IgM also recognize PC-related determinants expressed on apoptotic cells¹⁴ but it has not been reported whether passive transfer of these human IgM antibodies can affect in vivo apoptotic clearance or conveys or alters immunologic properties.

The naturally occurring or wildtype murine T15 antibody, while originally reported as an IgA antibody produced by a plasmacytoma, was later reported to be a natural IgM antibody (i.e., a type of antibody produced in many murine strains without prior immunization or demonstrable intentional immune exposure), and recognizes a determinant now known to be specific for dead and dying cells, including apoptotic cells⁶. The murine T15 antibody was first reported to be specific for the immunodominant PC moiety in the teichoic acid cell wall polysaccharide (C-PS) of pneumococci¹⁶. While T15 antibodies bind PC as a hapten, they do not bind to reduced native phospholipids, such as phosphatidyl choline (PtC), even though these neutral phospholipids contain the PC group. However, the prototypic T15 antibody (that expresses specific T15H:κ22L paired) rearrangements are devoid of “N” (non-templated) insertions at splice sites, which suggests they arise in early development, as perinatal liver B cells do not express terminal deoxytransferase (TdT) responsible for N insertions¹⁷, and are later exclusively represented in the B-1 cell compartment. In most adult mice, PC-responsive B cells that express the T15 clono-specific markers dominate all anti-PC responses, including those to pneumococci¹⁸. Of all anti-PC antibodies, T15 antibody¹⁹⁻²¹ provide the most protection from systemic pneumococcal infection from experimental strains²², due to their efficient clearance of these microbes from the blood²³.

The T15 antibody has also been shown to recognize and bind PC expressed on oxidatively modified low density lipoprotein (OxLDL) that contains phospholipids^{6;24} as illustrated in FIG. 1. While macrophages can recognize, take up and clear the OxLDL, studies using an in vitro system have been reported to show that the addition of the EO6 antibody, which is encoded by the same variable region genes as the T15 IgM, blocked the in vitro uptake and clearance of apoptotic cells²⁵. This data suggested a very different activity is induced by interactions of phagocytic cells with a T15 IgM anti-PC antibody and functionally equivalent antibodies, than is described in the compositions described herein. This data also does not predict how, by affecting the clearance activities of a phagocytic cell, therapeutic benefits could be imparted in other inflammatory or autoimmune diseases.

Present treatments for inflammatory disorders and/or autoimmune diseases include administering immunosuppressive drugs like corticosteroids and drugs such as methotrexate, cyclophosphamide, azathioprine, cyclosporin A, sulfasalazine, hydroxychloroquine, leflunomide. These immunosuppressive drugs affect many cells in the body in addition to the cells of the immune system of the subject. In addition, treatments have also been developed that are specific cytokine blocking agents such as infliximab, etanercept, kinaret or other cytokine blockers or antagonists. Long-term use of either type of agent can increase the risk of infection and in some cases increase the risk for developing cancer. Moreover, these drugs merely slow down the progress of the disorder, which usually resumes after the therapy is discontinued. Additionally, prolonged therapy with these nonspecific drugs often produces toxic side effects, including kidney failure, bone marrow suppression, pulmonary fibrosis, diabetes, and liver function disorders. These drugs may also gradually lose effectiveness after months to years of use.

Alternatively, therapeutic agents that are anti-inflammatory drugs have been used which include Non-Steroidal Anti-Inflammatory Drugs (NSAIDS) as well as corticosteroid compounds such as prednisone and methylprednisolone. However, these steroids also have significant toxic side effects associated with their long-term use.

Thus, current treatments for inflammatory disorders and/or autoimmune diseases often display limited efficacy, involve significant toxic side effects, and in many cases cannot be used continuously for prolonged periods of time.

Accordingly, there exists a need for treatments that are effective and more potent for treating such autoimmune diseases and inflammatory diseases, and great benefits could result from administration of an agent that interrupts key pathogenetic pathways.

### SUMMARY OF THE INVENTION

The invention provides methods for inhibiting autoimmunity or autoimmune disease in a subject which comprises administering a binding protein (e.g., a recombinant protein or antibody or fragment or variant thereof) that specifically recognizes and binds dead or dying cells through interactions with an antigen exposed on the dead or dying cells, where the antigen is selected from the group consisting of a phosphorylcholine (PC) determinant, a phosphatidyl serine (PS) determinant, a malondialdehyde (MDA) determinant, linoleic acid peroxide, malondialdehyde and 4-hydroxynonenal and cardiolipin in the subject, thereby affecting the efficiency for apoptotic clearance and/or affecting the capacity of macrophages and/or dendritic cells to induce or modulate or downgrade disease associated immune and/or inflammatory response states. In another embodiment, the method comprises contacting dead or dying cells exposing antigens selected from the group consisting of a phosphoryl choline (PC) determinant, a phosphatidyl serine (PS) determinant, MDA, and cardiolipin in the subject, with one, several or multiple binding proteins, such as antibodies that recognize and bind one or more of the antigens so that the dead or dying cells are effectively cleared from the subject thereby inhibiting autoimmunity, autoimmune disease and/or inflammatory disease in the subject.

The invention is directed to methods for ameliorating an autoimmune disease and/or inflammatory disease by treatment with variable antibody regions, or functionally equivalent agent, that recognizes and binds antigens exposed on the dying and dead cells in a subject.

The invention is also directed to methods for ameliorating an autoimmune disease or inflammatory disease with a recombinant protein, or antibody, that recognizes and binds an antigen exposed on the dead or dying cells, where the antigen is selected from the group consisting of phosphorylcholine, phosphatidyl serine, malondialdehyde, cardiolipin, or their metabolic products, or the metabolic products of other phospholipids on dead or dying cells

The invention additionally provides methods of alleviating signs and symptoms (e.g., undesirable side effects) associated with chemotherapy treatment (e.g. associated with impaired clearance of dead or dying cells), comprising administering to a patient a pharmaceutically effective amount of a T15 PC-binding protein such as an antibody (including fragment or variant thereof), or a recombinant protein, so as to inhibit inflammation, and thereby alleviate symptoms (e.g., undesirable side effects) associated with chemotherapy.

In addition, the invention provides binding proteins such as antibodies or recombinant proteins for example, antibodies or recombinant proteins containing all or part of the antigen-binding region of a T15 antibody. In one embodiment, the recombinant protein is a human/murine recombinant antibody, the antigen-binding region of which competitively inhibits the immunospecific binding of the T15 antibody to its target antigen, or which has in vitro binding specificity and activity for antigens expressed on dead and/or dying cells. Pharmaceutical compositions and kits are also provided.

### BRIEF DESCRIPTION OF THE FIGURES

FIG. 1 is a depiction of a phosphoryl choline (PC) antigenic triad. PC is displayed on microbial pathogens, atherosclerosis associated plaques and oxidatively modified LDL, and dead and dying cells, which enables these substances to be recognized by cell receptors, B cell antigen receptors and antibodies.

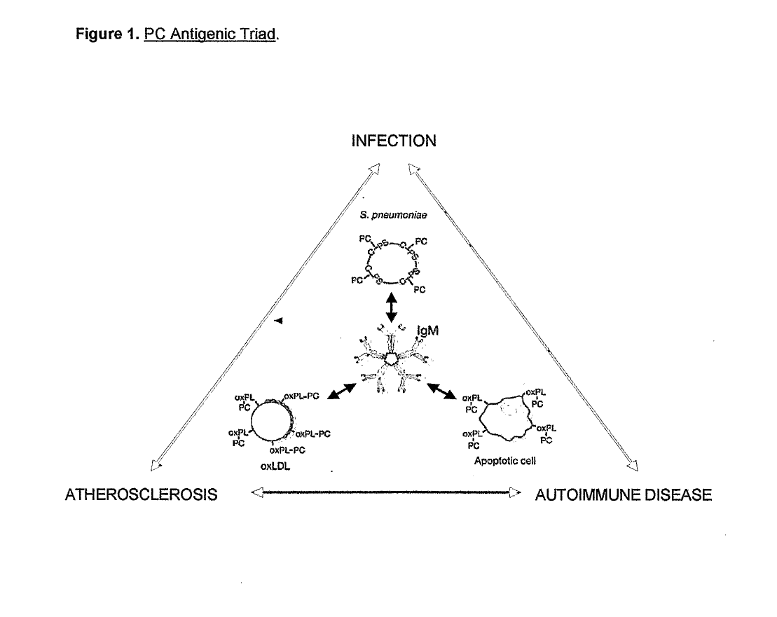

FIG. 2 is a depiction of how T15 IgM recognizes apoptosis-associated neo-epitopes to facilitate packaging and complement factor deposition on dead and dying cells, apoptotic blebs and cell debris.

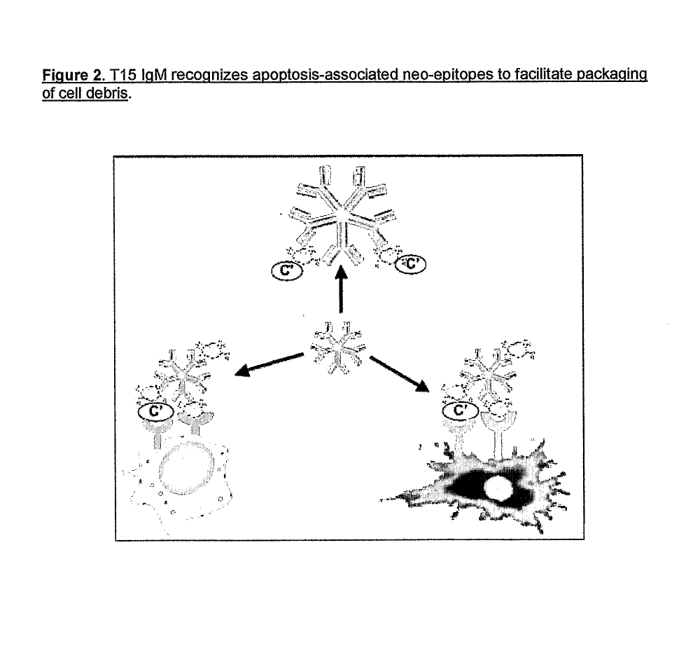

FIG. 3 demonstrates the increased survival of (NZWxBXSB)F1 mice following treatment with the T15 IgM anti-PC antibody, as described in detail in Example 1, infra.

FIG. 4 illustrates a flow cytometric assay in which the T15 IgM anti-PC antibody is shown to induce in vitro complement deposition on apoptotic thymocytes, an example of a representative dying cell, as described in detail in Example 2, infra.

FIG. 5 shows that the T15 anti-PC antibody enhances in vivo phagocytosis by peritoneal macrophage (Mφ) of apoptotic thymocytes, as described in detail in Example 2, infra.

FIG. 6 illustrates the outcome of treatment of muMT−/− mice, which are B-cell and antibody deficient mice, as treatment with T15 IgM but not by an IgM isotype control of irrelevant binding specificity, NC17-D8, enhances the in vivo clearance of apoptotic thymocytes, as described in detail in Example 2, infra.

FIG. 7 demonstrates that T15 IgM treatment blunts responses to agonists for Toll-like receptors (TLR), as described in detail in Example 2, infra.

FIG. 8 shows that T15 IgM treatment depletes splenic marginal zone (MZ) B cells and transitional B-cell precursors, as described in detail in Example 2, infra.

FIG. 9 demonstrates that IgG anti-PC antibodies, generated by expression of T15 antibody genes using the pIGG expression vector, display binding activity for PC with high affinity by a solid phase immunoassay, as described in detail in Example 3, infra.

FIG. 10 shows IgG anti-PC antibody, generated by expression from the pIGG vector display, bind dead and dying cells with high affinity, as shown in flow cytometris assays, as described in detail in Example 3, infra.

FIG. 11 demonstrates T15 IgM treatment decreases levels of transcripts of inflammation associated cytokines and factors, as detected by quantitative Taqman assay, and described in detail in Example 4, infra.

### DETAILED DESCRIPTION OF THE INVENTION

### Methods and Compositions of the Invention

### Methods

The invention provides methods for inhibiting an immune response (e.g., an autoimmune response) and/or immune system diseases (e.g., autoimmune disease, pathologic autoimmune disease or condition and/or inflammatory disease) in a subject, associated with dead or dying cells that expose an antigen, using binding proteins or small molecules that recognize the antigen. In one embodiment, the method comprises contacting a binding protein (e.g., an antibody) that recognizes and binds an antigen, which is or comprises an exposed phospholipid, or a phospholipid derived antigen, that is exposed on dead or dying cells, which results in the inhibition of an immune response (e.g., a response involving autoimmunity or inflammatory disease or autoimmune disease) in the subject. In another embodiment, the method comprises contacting dead or dying cells exposing antigens comprising any of a phosphorylcholine (PC) determinant, phosphatidyl serine (PS) determinant, MDA, and/or cardiolipin with multiple binding proteins (e.g., antibodies) that recognize and bind the multiple antigens that are exposed on the dead or dying cells, so that the dead or dying cells are cleared more rapidly and/or effectively from the subject thereby inhibiting autoimmunity or autoimmune disease or inflammatory disease in the subject.

As used herein a “pathologic autoimmune response” includes a clinical state in which a subject's lymphocytes recognize components of the body (i.e., B cell and/or T cell autoreactivity) in a setting of inflammation and/or tissue injury and damage.

As used herein, “binding protein” means a molecule that recognizes the exposed antigen on the dead or dying cells. Binding protein includes any substance that contains a region that recognizes the exposed antigen and includes, but is not limited to, antibodies of the invention (including antibody fragments or variants), recombinant proteins thereof, and small molecules thereof.

As used herein, this “inhibition” means to interfere with the activation of the receptor, signaling pathway or molecule essential for an inflammatory response, as detected by an art-recognized test (for example, the joint swelling and leukocyte infiltration into the joints as is induced in the collagen induced arthritis model). Inhibition may be partial or total. In another example, inhibition of an inflammatory response can be detected by determining reduction of inflammatory factors and mediators, such as IFN type I and type II, and related cytokines like IL-12. Inhibition may be partial or total.

As used herein, reducing the symptoms of auto immunity or inflammatory response means to improve the symptoms of auto immunity or inflammatory response, e.g., by enhancing clearance of dead or dying cells that expose an antigen that is essential for the auto immunity or inflammatory response to occur. Symptoms include: joint swelling and leukocyte infiltration into joints (arthritis), reduction of factors and mediators associated with auto immunity or inflammatory response, such as IFN type I and type II, and cytokines including IL-12, and interferon-γ although the specific cytokines that are overexpressed differs between diseases. In addition, although not wishing to be bound by any theory, the methods and compositions of the invention inhibit or reduce the symptoms of autoimmunity or inflammatory disease in a subject by altering the responses of leukocytes capable of interacting with dead or dying cells from the subject.

In accordance with the practice of this invention, “dead or dying cells,” includes cell death or dying by any pathway, included programmed cell death (e.g., through apoptosis), autophagy or non-programmed cell death (e.g., by necrosis or injury).

In accordance with the practice of the invention, the antigen that is exposed on the dead or dying cells is an antigen that is newly exposed, i.e., exposed only when the cell is dead or dying. In a healthy cell, although this antigen may also be present in the cell membrane it is cryptic or sequestered such that an antibody (e.g., a T15 antibody) does not bind, or exhibits reduced binding to, these cells.

The antigen on these dead or dying cell may comprises any of a phosphoryl choline (PC) determinant⁶, phosphatidyl serine (PS) determinant²⁶, MDA determinant²⁵, and cardiolipin^{27;28}. Other functionally equivalent antigens or determinants, that are expressed on dead and dying cells, may be targeted by an antibody or recombinant protein, generated according to this invention, even when the identity of the antigen or determinant is not known.

In accord with the practice of the invention, the antigen, which is an exposed phospholipid, or phospholipid derived antigen, that is exposed on the dead or dying cells includes, but is not limited to, an antigen comprising any of phosphorylcholine (PC) determinant, phosphatidyl serine (PS) determinant, MDA determinant, and cardiolipin.

In one embodiment of the methods, the antibody is an antibody that recognizes and binds phosphorylcholine (PC) determinants exposed on the dead or dying cell. For example, the antibody that recognizes and binds the PC determinant may be a T15 antibody or variant or fragment thereof, or a functionally equivalent protein. Additional examples of suitable antibodies or variant or fragment thereof for use in the methods of the invention may be found, supra, in the section entitled “Compositions of the Invention.”

As used herein, “T15 antibody or variant or fragment thereof” means the T15 antibody, or any antibody or variant or fragment thereof that comprise the same or closely related variable regions of the T15 antibody as described by Shaw et al.⁶. A variant is a molecule that shares sequence similarity and activity of its parent molecule. For example, a variant of T15 antibody includes a molecule having an amino acid sequence at least 80% similar to the variable domain of T15 antibody, encoded by the S107.1 heavy chain variable region gene and which recognizes and binds PC and/or other phospholipid derived determinant. A variant means any change to the amino acid sequence and/or chemical quality, of the amino acid e.g., amino acid analogs, from that encoded by the T15 sequence. The antibody can be polyclonal, monoclonal, chimeric, or humanized antibodies.

In one embodiment, the T15 antibody fragment can be a T15 Fab molecule. In another, the T15 antibody fragment can be a T15 F(ab′)₂ molecule. Further still, the T15 antibody fragment can be a T15 Fv molecule. Additionally, in another example, the T15 antibody fragment is a T15 single chain Fv molecule.

The invention further provides methods for inhibiting an inflammatory response (e.g., by reducing the symptoms of an inflammatory response) in a subject with a binding protein (e.g., an antibody or recombinant protein) that contacts dead or dying cells, which have an exposed antigen comprising any of phosphorylcholine (PC) determinant, phosphatidyl serine (PS) determinant, MDA determinant, and cardiolipin in the subject. In a further embodiment, the method comprises contacting dead or dying cells having one or more exposed antigens comprising any of a phosphorylcholine (PC) determinant, a phosphatidyl serine (PS) determinant, malondialdehyde (MDA) determinant, and cardiolipin, with an antibodies (including fragments or variants thereof) that recognizes and binds one or more of the antigens that is exposed on the dead or dying cells, thereby inhibiting the inflammatory response in the subject.

As used herein, “inflammatory response” means the recruitment of cells to sites of injury or immunization or immune response in a subject, with release of inflammatory mediators that can include, but are not limited to, certain types of cytokines, histamine, chemokines, prostaglandins and others. Inflammation is defined by the presence of tissue destruction and release of destructive cytokines and chemokines, and abnormal tissue accumulation of cells of the innate and adaptive immune systems.

Additionally, the invention provides methods of alleviating signs, symptoms and/or complications associated with cancer or chemotherapy by administering to a subject having cancer and/or undergoing chemotherapy a pharmaceutically effective amount of a binding protein (e.g., an antibody or recombinant protein of the invention) so as to inhibit inflammation and thereby alleviating signs, symptoms and/or complications associated with chemotherapy.

As used herein, an “effective amount” of a composition of the invention (e.g. a binding protein) is defined as an amount that inhibits or reduces inflammation (e.g., reduces the symptoms of inflammation) and/or tissue damage and destruction. For example, an effective amount of a T15 antibody, may be defined as the amount of the binding protein (e.g., antibody) that, when bound to dead or dying cells, promotes removal of the dead or dying cells, from the injured area or elsewhere in the body. These binding proteins may also induce macrophages or dendritic cells or other leukocytes to express pro-inflammatory surface molecules or release of cytokines or other soluble factors.

The most effective mode of administration and dosage regimen for the compositions of the invention (e.g., binding proteins such as antibodies or recombinant proteins) depends upon the location, extent, or type of the disease being treated, the severity and course of the medical disorder, the subject's health and response to treatment and the judgment of the treating physician. Accordingly, the dosages of the compositions of the invention should be titrated to the individual subject and/or by the specific medical condition or disease.

By way of example, the interrelationship of dosages for animals of various sizes and species and humans based on mg/m² of surface area is described by Freireich, E. J., et al.²⁹. Adjustments in the dosage regimen may be made to optimize suppression or modulation of the immune response responsible for disease or after transplantation for graft rejection, e.g., doses may be divided and administered on a daily basis or weekly or biweekly or monthly basis or the dose reduced proportionally depending upon the situation (e.g., several divided doses may be administered daily or proportionally reduced depending on the specific therapeutic situation).

It would be clear that the dose of the composition of the invention required to achieve an appropriate clinical outcome may be further reduced with schedule optimization.

The invention further provides methods for reducing the symptoms of an immune system disease (e.g., an autoimmune disease or inflammatory disease) with a binding protein, such as an antibody, that specifically recognizes and binds an antigen exposed on dead or dying cells.

The invention further provides methods for ameliorating autoimmune disease states (e.g., reducing the symptoms thereof) which comprises administering a binding protein (e.g., an antibody or recombinant protein) to a subject, which protein specifically recognizes and binds dead or dying cells. In accord with the practice of the method, the recombinant protein can comprise antibody variable regions, or portions thereof. In accord with the method the antibody comprises variable antibody regions or portions thereof that recognize and bind phosphorylcholine, phosphatidyl serine, malondialdehyde, cardiolipin, or their metabolic products, or the metabolic products of other phospholipids exposed on dead or dying cells.

As used herein, “ameliorating” a disease means to obtain improvement, for example, by managing a disease by medicinal or other therapies.

“Treatment” of a disease may ameliorate the symptoms of a disease, reduce the severity of a disease, alter the course of disease progression and/or ameliorate or cure the basic disease problem. For example, to treat an autoimmune disease may be accomplished by regulating, modulating or suppressing an immune response. Alternatively, treating an auto-immune disease may be accomplished by preventing the disease from occurring or progressing through the use of drugs such as the compositions of the invention, described herein.

In one embodiment, the methods for ameliorating an inflammatory disease (e.g., reducing the symptoms thereof) comprises administering to a subject a binding protein (such as an antibody or antibodies of the invention or recombinant protein) that recognizes and binds dead or dying cells. In accord with the practice of the method, the recombinant protein comprises antibody variable regions, or portions thereof that recognizes a phospholipid antigen. Further, in accord with the practice of the method, the antibody recognizes and binds dead or dying cells because of newly exposed antigens thereon which antigens include, but are not limited to, phosphorylcholine, phosphatidyl serine, malondialdehyde, cardiolipin, or their metabolic products, or the metabolic products of other phospholipids. In a preferred embodiment, the antibody is an antibody that recognizes and binds a phosphoryl choline determinant exposed on the dead or dying cell.

The invention also encompasses methods for ameliorating autoimmune disease states (e.g., reducing symptoms thereof), which may be associated with impaired clearance of dead or dying cells or their breakdown products (e.g., apoptotic blebs) in a subject, with a binding protein that contacts dead or dying cells exposing antigens comprising any of a phosphorylcholine determinant, phosphatidyl serine, malondialdehyde, cardiolipin, or their metabolic products and/or other phospholipid moieties. In one embodiment, the binding protein is one or more antibodies that recognize and bind one or more antigens that are exposed on the dead or dying cells, so that there is improved clearance of dead or dying cells from the subject or inhibition or modulation of leukocyte activation or cytokine production in the subject. The leukocytes include, but are not limited to lymphocytes, dendritic cells and/or macrophages and/or other phagocytic cells.

The methods and compositions of the invention can be used to inhibit an immune system disease or reduce the symptoms of immune system disease that results, for example, from autoimmunity or inflammatory responses associated with reduced clearance of dead or dying cells in a subject. An example of an immune system disease is lupus erythematosus or autoimmune nephritis. In another example, the immune system disease is an autoimmune disease selected from a group consisting of psoriasis, lymphocytic angiitis, Hashimoto's thyroiditis, primary myxedema, Graves' disease, pernicious anemia, autoimmune atrophic gastritis, Addison's disease, diabetes mellitus, Goodpasture's Syndrome, myaesthenia gravis, pemphigus, Crohn's disease, sympathetic ophthalmia, autoimmune uveitis, multiple sclerosis, autoimmune hemolytic anemia, idiopathic thrombocytopenia, primary biliary cirrhosis, chronic active hepatitis, ulcerative colitis, Sjogren's syndrome, inflammatory arthritis, polymyositis, and scleroderma. Arthritis can include psoriatic arthritis or rheumatoid arthritis or Reiter's syndrome. Additionally, the inflammatory system disease can be atherosclerotic vascular disease. In another embodiment, the immune system disease or inflammatory disease is macular degeneration.

Further, the immune system disease or inflammatory disease can be a disease or pathologic condition associated with organ or bone marrow transplantation (e.g., cells, tissues, or organs). In one embodiment, the transplantation associated disease is graft versus host disease (GVHD). In another example, the transplantation disease is transplant rejection such as renal, cardiac, lung or liver allograft transplant rejection.

The invention additionally includes methods of treating cancer or alleviating signs or symptoms associated with cancer comprising administering to a patient a pharmaceutically effective amount of a binding protein (e.g., an antibody to PC (such as a T15 antibody) or other phospholipid associated determinant) so as to inhibit inflammation associated with impaired clearance of dead and dying cells, or affecting the responses of leukocytes and thereby treating cancer or alleviating signs or symptoms associated with cancer.

Additionally the invention provides methods for reducing levels of circulating autoantibodies in a subject comprising administering to the subject an antibody directed against an antigen comprising any of a phosphorylcholine (PC) determinant, phosphatidyl serine determinant, MDA determinant, linoleic acid peroxide determinant, 4-hydroxynonenal determinant, and cardiolipin determinant, which antigen is exposed on the dead or dying cells or their breakdown products thereby reducing levels of circulating autoantibodies or inflammation in the subject.

In accordance with the practice of the invention, the methods of the invention can further comprise, in addition to administering one or more of the binding proteins (e.g., antibodies) described above, administration of one or more therapeutic agents to further inhibit an immune system disease (such as autoimmunity and/or inflammatory responses), treat cancer, or to reduce the undesirable symptoms associated with chemotherapy. These agents include, but are not limited to, steroids, glucocorticoids, drug toxins, alkylating agents, anti-neoplastic drugs, enzymes, antibodies, conjugates, immunosuppressive drugs like corticosteroids and drugs such as methotrexate, cyclophosphamide, azathioprine, cyclosporin A, sulfasalazine, hydroxychloroquine, leflunomide. These immunosuppressive drugs affect many cells in the body in addition to the cells of the immune system of the subject. In addition, treatments have also been developed that are specific cytokine blocking agents such as infliximab, etanercept, kinaret or other cytokine blockers or antagonists, a molecule that blocks TNF receptors (e.g., pegsunercept), a molecule that blocks cytokine function (e.g., AMG719), a molecule that blocks LFA-1 function (e.g., efalizumab). Also, anti-inflammatory agents like acetyl salicylic acid, choline magnesium salicylate, diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, other nonspecific COX inhibitors and Cox-2 specific inhibitors, meloxicam, as well as other analgesics such as codeine phosphate, propoxyphene napsylate, oxycodone hydrochloride, oxycodone bitartrate, tramadol.

It would be clear to one skilled in the art that dosage of these therapeutic agents will vary depending on the particular therapeutic agents being used. Specific examples of appropriate dosages, depending on the therapeutic agents, are described below.

An effective amount of a composition of the invention can be in a range of about 0.1 mg/week to 40 mg/week; 0.1 mg/week to 5 mg/week; 5 mg/week to 10 mg/week; 10 mg/week to 30 mg/week; 30 mg/week to 35 mg/week; 0.1 mg/week to 100 mg/week; or mg/week to 50 mg/week. In another embodiment, a composition of the invention can be administered in an amount of about 50 mg/week or 25 mg twice weekly. It would be clear to one skilled in the art that dosage range will vary depending on the disease being treated and the co-treatment agent being used.

Methotrexate is an anti-metabolite molecule that interferes with DNA synthesis, purine metabolism, repair and cellular replication. Methotrexate functions as an inhibitor of dihydrofolic acid reductase i.e. it is a folic acid antagonist. Methotrexate is commonly administered in an amount about 0.1 to 40 mg per week with a common dosage ranging about 5 to 30 mg per week. Methotrexate may be administered to a subject in various increments: about 0.1 to 5 mg/week, about 5 to 10 mg/week, about 10 to 15 mg/week, about 15 to 20 mg/week, about 20 to 25 mg/week, about 25 to 30 mg/week, about 30 to 35 mg/week, or about 35 to 40 mg/week. In one embodiment, an effective amount of a composition of the invention and a cotreatment agent such as methotrexate, is an amount about 10 to 30 mg/week.

Cyclophosphamide, an alkylating agent, may be administered in dosages ranging about 1 to 10 mg/kg body weight per day.

Cyclosporine (e.g. NEORAL^{R}) also known as Cyclosporin A, is commonly administered in dosages ranging from about 1 to 10 mg/kg body weight per day. Dosages ranging about 2.5 to 4 mg per body weight per day are commonly used.

Chloroquine or hydroxychloroquine (e.g. PLAQUENIL^{R}), is commonly administered in dosages ranging about 100 to 1000 mg daily. Preferred dosages range about 200-600 mg administered daily.

Sulfasalazine (e.g., AZULFIDINE EN-tabs^{R}) is commonly administered in amounts ranging about 50 to 5000 mg per day, with a common dosage of about 2000 to 3000 mg per day for adults. Dosages for children are commonly about 5 to 100 mg/kg of body weight, up to 2 grams per day.

Gold salts are formulated for two types of administration: injection or oral. Injectable gold salts are commonly prescribed in dosages about 5 to 100 mg doses every two to four weeks. Orally administered gold salts are commonly prescribed in doses ranging about 1 to 10 mg per day.

D-penicillamine or penicillamine (CUPRIMINE^{R}) is commonly administered in dosages about 50 to 2000 mg per day, with preferred dosages about 125 mg per day up to 1500 mg per day.

Azathioprine is commonly administered in dosages of about 10 to 250 mg per day. Preferred dosages range about 25 to 200 mg per day.

Anakinra (e.g. KINERET^{R}) is an interleukin-1 receptor antagonist. A common dosage range for anakinra is about 10 to 250 mg per day, with a recommended dosage of about 100 mg per day.

Infliximab (REMICADE^{R}) is a chimeric monoclonal antibody that binds to tumor necrosis factor alpha (TNFα) and inhibits the activity of TNFα. Infliximab is commonly administered in dosages about 1 to 20 mg/kg body weight every four to eight weeks. Dosages of about 3 to 10 mg/kg body weight may be administered every four to eight weeks depending on the subject.

Etanercept (e.g. ENBREL^{R}) is a dimeric fusion protein that binds the tumor necrosis factor (TNF) and blocks its interactions with TNF receptors. Commonly administered dosages of etanercept are about 10 to 100 mg per week for adults with a preferred dosage of about 50 mg per week. Dosages for juvenile subjects range about 0.1 to 50 mg/kg body weight per week with a maximum of about 50 mg per week. For adult patients, etanercept is commonly administered e.g., injected, in 25 mg doses twice weekly e.g., 72-96 hours apart in time.

Leflunomide (ARAVA^{R}) is commonly administered at dosages about 1 and 100 mg per day. A common daily dosage is about 10 to 20 mg per day.

### Compositions

### Binding Proteins and Small Molecules of the Invention

The invention further provides binding proteins and small molecules that bind to antigens comprising phospholipids associated determinants that are exposed on dead or dying cells, or their breakdown products (such as apoptotic blebs). These determinants include, but are not limited to a phosphorylcholine determinant, a phosphatidyl serine determinant, MDA determinant, linoleic acid peroxide determinant, 4-hydroxynonenal determinant, and cardiolipin determinant or other metabolic products of neutral phospholipids moieties.

Binding proteins can include antibodies such as polyclonal, monoclonal, chimeric, humanized and/or other form of recombinant antibodies. Antibodies can be from any source, e.g., rat, dog, cat, pig, horse, mouse or human. Further, antibodies of the invention may have immunoglobulin constant regions of the following isotypes IgM, IgA, IgG, IgE, or IgD.

The antigens exposed on the dead or dying cells comprise any of a phosphorylcholine determinant, a phosphatidyl serine determinant, MDA determinant, linoleic acid peroxide determinant, 4-hydroxynonenal determinant, and cardiolipin determinant or other metabolic products of neutral phospholipids moieties. A preferred example of an antigen having a phosphorylcholine determinant is the antigen recognized by a T15 antibody.

The present invention includes the discovery of unexpected functional roles for PC, a molecular epitope long known to decorate many common microbial pathogens for immune recognition, which also mark dead or dying cells and oxidatively modified low density lipoprotein (OxLDL)²⁴ that is a major mediator of atherosclerosis. Atherosclerosis is believed to result from aberrant clearance of oxidatively modified phospholipid debris, and many of the same pathways for apoptotic recognition and clearance are also involved in the development of atherosclerotic lesions. There is extensive documentation that both animal models and clinical disease of atherogenesis are associated with limited autoimmune responses to OxLDL. To investigate the potential functional roles of anti-OxLDL antibodies, a panel of splenic B-cell lines specific for OxLDL were isolated from atherosclerosis-prone hyperlipidemic Apolipoprotein-E deficient mice. The prototype of this group was termed E06, which is an IgM with antibody genes expressed by this independently isolated OxLDL-specific clone, with the exact same canonical antibody gene rearrangements, without somatic hyper mutation, that was first described decades earlier for the classical T15 anti-PC B-cell clone²⁴.

Preferred antibodies will selectively bind to the antigens above (e.g., a PC antigen) and will not bind (or will bind weakly) to determinants accessible on the surface of healthy cells. The most preferred antibodies will specifically bind to PC and functionally equivalent antigens. It is intended that the term “specifically bind” means that the antibody predominantly binds to a phospholipid antigen above (e.g., a PC antigen). Antibodies (e.g., T15 antibodies) that are particularly contemplated include monoclonal and polyclonal antibodies as well as fragments thereof (e.g., recombinant proteins) containing the antigen binding domain and/or one or more complementarily determining regions of these antibodies. These antibodies can be from any source, e.g., rat, dog, cat, pig, horse, mouse or human.

In one embodiment, the T15 antibody specifically binds to the extra cellular surface of an exposed phospholipid determinant, e.g., on the cell surface of dead or dying cells. It is intended that the term “extra cellular surface” means any portion of a phospholipid or phospholipid conjugate that is on the exterior to the membrane of the cell. As will be understood by those skilled in the art, the regions or epitopes of this phospholipid, or functionally equivalent determinant to which an antibody is directed may vary with different antibodies and the intended application. For example, antibodies intended for use in an immunoassay for the detection of membrane-bound phospholipid determinants on dead or dying cells should be directed to an accessible epitope on the membrane-bound phospholipid or phospholipids conjugate. Such antibodies are described in the Examples which follow.

The binding proteins of the invention also encompasses antibody fragments that specifically recognize phospholipid determinant associated with carbohydrates or proteins (e.g., PC antigen). As used herein, an antibody fragment is defined as at least a portion of the variable region of the immunoglobulin molecule that binds to its target, i.e., the antigen binding region. Some of the constant region of the immunoglobulin may be included. In accordance with the practice of the invention, the constant region of the immunoglobulin may be any of the following isotypes, namely, IgM, IgA, IgG, IgE, or IgD or portion(s) thereof. IgM and IgG constant regions are preferred.

Antibody variants (e.g., T15 antibody variants) are also contemplated by the invention. While the antigen (Ag) binding specificity of an antibody is defined by the variable region contributions of the Fab portion, the constant regions elicit four main effectors functions: antibody dependent cellular cytotoxicity (ADCC); phagocytosis; complement dependent cytotoxicity (CDC); and half-life/clearance rate. ADCC and phagocytosis are mediated through complexes of cells or cell products bound with antibody that interact with FcγR, while CDC is mediated by interactions with the complement cascade that begins with C1q deposition. The in vivo half-life of an IgG is mediated by binding of free antibodies to the neonatal Fc receptor (FcRn).

T15 antibody variants of the invention preserve binding activity for apoptotic cells and the capacity to activate complement essential for apoptotic clearance and tolerizing properties. In one embodiment, the T15 antibody variant is a T15 antibody having an IgM isotype. In another embodiment, the T15 antibody variant is a T15 antibody having an IgG isotype (a T15 IgG variant) that retain the capacity to interact with the FcRn responsible for the recycling of trancytosed antibody that increases in vivo half-life.

For example, the expression of PC antigen newly exposed on both dead and dying cells (i.e., exposed upon death or near death), and the cell surface location of PC, represent characteristics of a preferred marker for screening, diagnosis, prognosis, and follow-up assays and imaging methods. In addition, these characteristics indicate that the PC antigen may be a target for therapeutic methods such as targeted antibody therapy, immunotherapy, and gene therapy. For example, the antibodies of the invention specifically recognize apoptotic cells in flow cytometric assays^{6;30}. In one embodiment, a panel of IgG T15 antibodies with identical variable regions, but with different gamma constant regions can be used to convey different Fcγ mediated effector properties. These antibodies can be further tested in in vitro assays, and their impact on in vivo autoimmune pathogenesis studied.

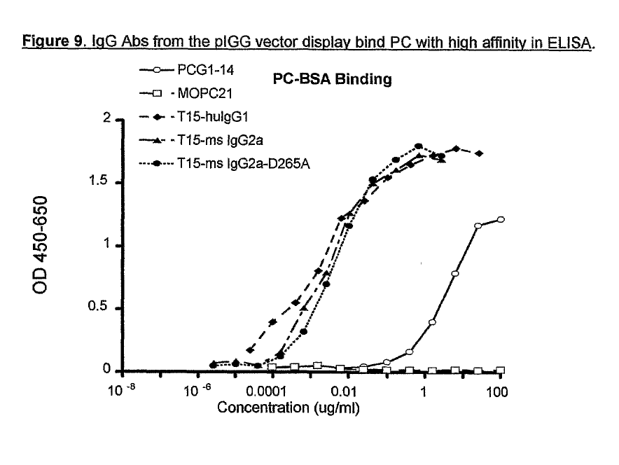

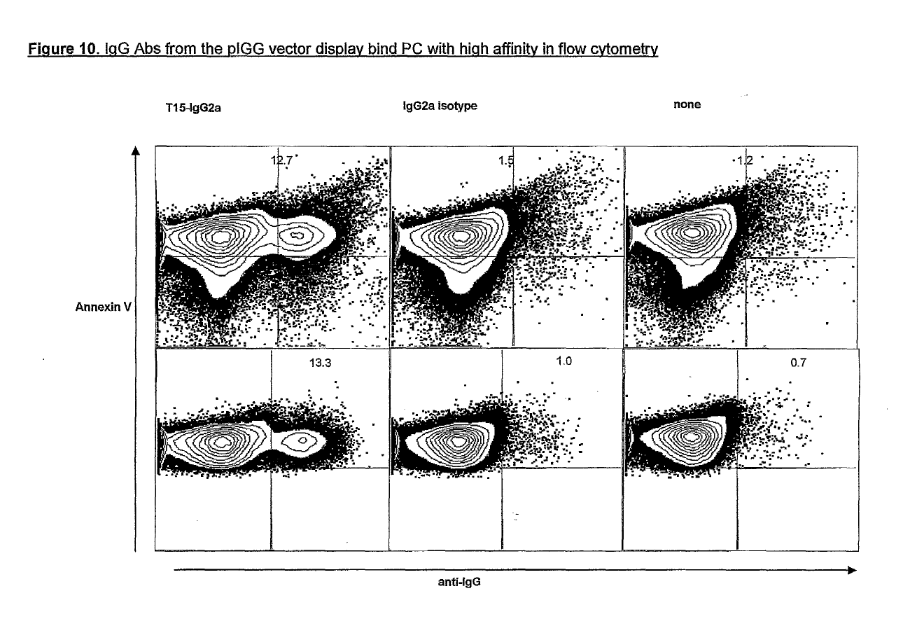

With the pIGG expression vector, a series of T15-expressing recombinant IgG antibodies has been generated. As the pIGG vector was provided with the cloned genes for a human IgG1 antibody³¹, the vector of the murine L chain gene (T15 VL-murine CK), and the T15 VH region, was subcloned into the pIGG vector and transfection studies yielded substantial T15 human chimeric IgG1 levels (˜2 ug/ml), and that expresses the T15 variable regions, which are recognized by the AB1-2 anti-idiotype that requires T15VH-T15VL regions for recognition³². This T15 IgG antibody expressed preserved efficient PC binding activity, comparable to the parental T15 mouse IgM, as judged by ELISA method that quantitatively measured the level of PC binding. In addition, swapping the DNA region that encodes for the heavy chain constant region for a γ chain, produce the T15 mouse (ms) IgG2a that was shown by ELISA to preserve PC binding reactivity, and which also recognizes dead and dying cells but not healthy cells, as demonstrated by flow cytometry (see FIGS. 9 and 10). By introducing a replacement mutation into the constant region the T15 mouse IgG2a D265A mutant was made that had fully preserved PC binding activity, but which had the predicted great impairment of such immune complexes with FcγRIII binding³³ as shown using an in vitro binding system with a recombinant hexahistidine tagged FcγRIII.

Binding proteins of the invention (e.g., T15 antibodies or other anti-PC antibodies or variants or fragments), may be particularly useful in diagnostic assays, imaging methodologies, and therapeutic methods in the management of autoimmune disease, inflammatory diseases or autoimmune disease states. Such assays can use one or more T15 antibodies or other antibody capable of recognizing and binding a PC antigen, or functionally equivalent determinant on dead and dying cells, and include various immunological assay formats well known in the art, including but not limited to, various types of precipitation, agglutination, complement fixation, radioimmunoassays (RIA), enzyme-linked immunosorbent assays (ELISA), enzyme-linked immunofluorescent assays (ELIFA), immunohistochemical analysis and the like. In addition, immunological imaging methods are also provided by the invention, including but limited to radioscintigraphic imaging methods using labeled T15 antibodies, or equivalent antibodies. Such assays may be clinically useful in the detection, monitoring, and prognosis of inflammatory disease, autoinmiune disease or inflammatory diseases associated with impaired removal of dying or dead cells.

T15 antibodies may also be used in methods for purifying PC and phospholipids determinants and for isolating antigenic homologues and related molecules. For example, in one embodiment, the method of purifying a PC antigen-protein determinant or PC-glycolipid conjugate comprises incubating a T15 antibody, which has been coupled to a solid matrix, with a lysate or other solution containing components of dead or dying cells under conditions which permit the T15 antibody, or functionally equivalent antibody, to bind the nominal antigen; washing the solid matrix to eliminate impurities; and eluting the binding protein from the coupled antibody. Additionally, T15 antibodies or anti-PC antibody may be used to isolate T15 positive dead or dying cells using cell sorting and purification techniques.

Other uses of the binding proteins of the invention (e.g., T15 antibodies) include generating anti-idiotypic antibodies that mimic the antigen or determinant of choice, e.g., a monoclonal anti-idiotypic antibody reactive with an idiotype on any of the monoclonal antibodies of the invention.

Various methods for the preparation of antibodies are well known in the art. For example, antibodies may be prepared by immunizing a suitable mammalian host using a desired cell, extract, or purified protein or phospholipid containing compound, peptide conjugate, or fragment, in isolated or immunoconjugated form (as described in³⁴). Cells expressing or overexpressing the desired antigen may also be used for immunizations. Similarly, any cell engineered to express the desired antigen may be used. This strategy may result in the production of monoclonal antibodies with enhanced capacities for recognizing endogenous antigen (e.g., PC antigen or functionally equivalent determinant) on dead or dying cells.

For example, using standard technologies and standard hybridoma protocols³⁴, hybridomas producing T15 monoclonal antibodies, monoclonal anti-PC antibodies or monoclonal antibodies to any defined phospholpid determinants may be generated. Even without prior knowledge of the molecular binding fine specificity, monoclonal antibodies specific for apoptotic but not healthy cells may also be generated.

Chimeric antibodies of the invention are immunoglobulin molecules that comprise a human and non-human portion. In one embodiment, the antigen combining region (e.g., variable region) of a chimeric antibody can be derived from a non-human source (e.g. murine) and the constant region of the chimeric antibody which confers biological effector function to the iminunoglobulin can be derived from a human source. The chimeric antibody should have the antigen binding specificity of the non-human antibody molecule and the effector function conferred by the human antibody molecule.

In general, the procedures used to produce chimeric antibodies can involve the following steps:
a) identifying and cloning the correct gene segment encoding the antigen binding portion of the antibody molecule; this gene segment (known as the VDJ, variable, diversity and joining regions for heavy chains or VJ, variable, joining regions for light chains or simply as the V or variable region) may be in either the cDNA or genomic form;
b) cloning the gene segments encoding the constant region or desired part thereof;
c) ligating the variable region with the constant region so that the complete chimeric antibody is encoded in a form that can be transcribed and translated;
d) ligating this construct into a vector containing a selectable marker and gene control regions such as promoters, enhancers and poly(A) signals;
e) amplifying this construct in bacteria (which may or may not be necessary);
f) introducing this DNA into eukaryotic cells (transfection) most often mammalian lymphocytes or other cell types (e.g. Chinese Hamster Ovary cells or T293 cells);
g) selecting for cells expressing the selectable marker or based on recombinant protein expression;
h) screening for cells expressing the desired chimeric antibody; and
i) testing the antibody for appropriate binding specificity and effector functions.

Antibodies of several distinct antigen binding specificities have been manipulated by these protocols to produce chimeric proteins. Likewise, several different effector functions have been achieved by linking new sequences to those encoding the antigen binding region. Some of these include enzymes³⁵, immunoglobulin constant regions from another species and constant regions of another immunoglobulin chain or with defined mutations^{36;37}. Muations or changes in the constant regions can also affect their effector functions. Additionally, procedures for modifying antibody molecules and for producing chimeric antibody molecules using homologous recombination to target gene modification have been described³⁸.

While the polyclonal antisera may be satisfactory for some applications, for pharmaceutical compositions, monoclonal antibody preparations are preferred. Immortalized cell lines which secrete a desired monoclonal antibody may be prepared using the standard method of Kohler and Milstein or modifications which effect immortalization of lymphocytes or spleen cells, as is generally known. Alternatively, a cell line is transfected with the vector that contains the antibody variable region and constant region genes. The immortalized cell lines secreting the desired antibodies are screened by immunoassay in which the antigen is PC or a functionally equivalent determinant. When the appropriate immortalized cell culture secreting the desired antibody is identified, the cells can be cultured either in vitro or by production in ascites fluid or in transgenic animals.

The desired monoclonal antibodies are then recovered from the culture supernatant or from the ascites supernatant or other fluids. Fragments of the monoclonal antibodies of the invention or the polyclonal antisera (e.g., Fab, F(ab′)₂, Fv fragments, fusion proteins) which contain the immunologically significant portion (i.e., a portion that recognizes and binds a PC determinant) can be used as antagonists, as well as the intact antibodies. Humanized antibodies directed against PC determinants are also useful. As used herein, a humanized T15 antibody is an immunoglobulin molecule which is capable of binding to PC on dead or dying cells and which comprises variable region framework (FR) regions having substantially the amino acid sequence of a human immunoglobulin and complementarity determining regions (CDRs) having substantially the amino acid sequence of non-human immunoglobulin or a sequence engineered to bind PC determinants. Methods for humanizing murine and other non-human antibodies by substituting one or more of the non-human antibody CDRs for corresponding human antibody sequences are well known (see for example,³⁹⁻⁴¹. See also,^{42;43}.

Use of immunologically reactive fragments, such as the Fab, Fab′, or F(ab′)₂ fragments is at times preferable, especially in a therapeutic context, as these fragments are generally less immunogenic than the whole immunoglobulin. Further, bi-specific antibodies specific and reactive for two or more epitopes may be generated using methods generally known in the art.

Further, antibody effector functions may be modified so as to enhance the therapeutic effect of the antibodies of the invention (e.g., T15 antibodies and anti-PC antibodies and antibodies to related phospholipid modified determinants). For example, cysteine residues may be engineered into the Fc region, permitting the formation of interchain disulfide bonds and the generation of homodimers which may have enhanced capacities for internalization, ADCC and/or complement-mediated cell killing, see for example,^{44;45}. Mutations in the constant regions may enhance complement activating activity and may impair interactions with FcγR that are undesirable, and mutations may improve in vivo half-life. Homodimeric antibodies may also be generated by cross-linking techniques known in the art (e.g.,⁴⁶). The invention also provides pharmaceutical compositions having the monoclonal antibodies or anti-idiotypic monoclonal antibodies of the invention.

The binding proteins, such as antibodies or fragments of antibodies, may also be produced, using current technology, by recombinant means. For example, regions that bind specifically to the desired regions of the PC determinant can also be produced in the context of chimeric or CDR grafted antibodies of multiple species origin. The invention includes an antibody, e.g., a monoclonal antibody which competitively inhibits the immunospecific binding of any of the monoclonal antibodies of the invention to PC determinant on dead or dying cells, and antibodies that display immunospecific binding for dead and dying cells through recognition and binding of other determinants on dead and dying cells but not healthy cells.

Alternatively, methods for producing fully human monoclonal antibodies, include phage display and transgenic methods, are known and may be used for the generation of human monoclonal antibodies (for review, see⁴⁷). For example, fully human anti-PC monoclonal antibodies, or functionally equivalent antibodies specific for PC determinants or other phospholipids determinants, may be generated using cloning technologies employing large human Ig gene combinatorial libraries (i.e., phage display)^{48;49}. Fully human anti-PC monoclonal antibodies may also be produced using transgenic mice engineered to contain human immunoglobulin gene loci⁵⁰.

Reactivity of anti-PC antibodies against the target antigen may be established by a number of well known means, including Western blot, immunoprecipitation, ELISA, and FACS analyses using, as appropriate, PC determinants, PC-containing phospholipids, PC-expressing dead or dying cells. Anti-PC antibodies may also be characterized in various in vitro assays, including complement-mediated tumor cell lysis, antibody-dependent cell cytotoxicity (ADCC), antibody-dependent macrophage-mediated cytotoxicity (ADMMC), tumor cell proliferation, etc.

The antibody or fragment thereof of the invention may be labeled with a detectable marker or conjugated to a second molecule, such as a therapeutic agent (e.g., a cytotoxic agent) thereby resulting in an immunoconjugate. For example, the therapeutic agent includes, but is not limited to, an anti-tumor drug, a toxin, a radioactive agent, a cytokine, a second antibody or an enzyme. Further, the invention provides an embodiment wherein the antibody of the invention is linked to an enzyme that converts a prodrug into a cytotoxic drug.

Examples of cytotoxic agents include, but are not limited to ricin, ricin A-chain, doxorubicin, daunorubicin, taxol, ethidium bromide, mitomycin, etoposide, tenoposide, vincristine, vinblastine, colchicine, dihydroxy anthracin dione, actinomycin D, diphteria toxin, Pseudomonas exotoxin (PE) A, PE40, abrin, arbrin A chain, modeccin A chain, alpha-sarcin, gelonin, mitogellin, retstrictocin, phenomycin, enomycin, curicin, crotin, calicheamicin, sapaonaria officinalis inhibitor, maytansinoids, and glucocorticoid and other chemotherapeutic agents, as well as radioisotopes such as ²¹²Bi, ¹³¹I, ¹³¹In, ⁹⁰Y, and ¹⁸⁶Re. Suitable detectable markers include, but are not limited to, a radioisotope, a fluorescent compound, a bioluminescent compound, chemiluminescent compound, a metal chelator or an enzyme. Antibodies may also be conjugated to an anti-cancer pro-drug activating enzyme capable of converting the pro-drug to its active form.

Techniques for conjugating or joining therapeutic agents to antibodies are well known (see, e.g.,⁵¹⁻⁵⁴.

Additionally, binding proteins of the invention include recombinant proteins. In one embodiment, the recombinant protein of the invention has an antigen-binding region of any of the antibodies of the invention and can be used to treat inflammatory diseases, autoimmune diseases or autoimmune disease states associated with impaired removal of dead or dying cells or reduce the symptoms thereof.

The invention also provides variants of the antibodies of the invention in the form of recombinant proteins. In one embodiment, the recombinant protein comprises the antigen-binding region of a T15 antibody. In another embodiment, the recombinant protein is a human/murine chimeric antibody. In yet another embodiment, the recombinant protein comprises an antigen-binding region which is joined to an IgG tail. Fab, F(ab′)₂, or Fv fragments of the recombinant proteins are encompassed by the invention. In another embodiment, it is any antibody specific for PC that is generated in the variations of forms described above. It may also be an antibody that is specific for any antigenic determinant that is preferentially expressed on dead and dying cells but not healthy cells.

Additionally encompassed by the invention is a bispecific antibody with a binding specificity for two different antigens. In one embodiment, one of the antigens is the PC antigen.

Additionally contemplated by the invention is a monoclonal antibody, the antigen-binding region of which competitively inhibits the immunospecific binding of the T15 antibody to its target antigen.

In one embodiment, one of the antigens is the PC antigen. Additionally encompassed by the invention is an antibody with binding that is specific for dead and dying cells and not healthy cells in an assay that demonstrates an activity functionally equivalent to that displayed by the T15 antibody, or other anti-PC antibody.

Further, the invention provides a human/murine recombinant antibody, the antigen-binding region of which competitively inhibits the immunospecific binding of the T15 antibody to its target antigen.

In one embodiment of the invention, the recombinant protein specific for apoptotic cells is conjugated to a therapeutic agent to form a recombinant protein conjugate. Examples of such therapeutic agents include, but are not limited to, an anti-tumor drug, a toxin, a radioactive agent, a second antibody or an enzyme.

The invention further provides a combination of an immunoconjugate comprising the recombinant protein linked to an enzyme capable of converting a prodrug into a cytotoxic drug, and said prodrug.

Further, the invention includes a composition comprising a combination of an immunoconjugate comprising the recombinant protein of the invention linked to an enzyme capable of converting a prodrug into a cytotoxic drug, and said prodrug.

A pharmaceutical composition useful in the treatment of autoimmunity or autoimmune disease, or inflammatory disease which in some cases is associated with impaired removal of dying or dead cells, comprising a pharmaceutically effective amount of the recombinant protein of the invention and an acceptable carrier. In one embodiment, the antibody is labeled with a label selected from the group consisting of a radiolabel, an enzyme, a chromophore, and a fluorochrome.

### Kits

In a further embodiment of the invention, the present invention provides kits (i.e., a packaged combination of reagents with instructions) containing the molecules of the invention useful for treating an autoimmune disease or inflammatory disease state or reducing the symptoms thereof.

The kit can contain a pharmaceutical composition that includes one or more binding proteins of the invention, for example, a soluble antibody of the invention alone (e.g., antibodies that recognize a common PC antigen) or combinations of such antibodies of the invention (e.g., antibodies that recognize multiple PC antigens or other phospholipid antigen or other apoptosis associated antigen), or with a second agent, and an acceptable carrier or adjuvant, e.g., pharmaceutically acceptable buffer, such as phosphate-buffered saline, Ringer's solution and dextrose solution. It may further include other materials desirable from a commercial and user standpoint, including other buffers, diluents, filters, needles, syringes, and package inserts with instructions for use. The agents may be provided as dry powders, usually lyophilized, including excipients that upon dissolving will provide a reagent solution having the appropriate concentration.

Second agents can include the following: steroids, glucocorticoids, drug toxins, alkylating agents, anti-neoplastic drugs, such as cyclophosphamide, enzymes, antibodies, conjugates, immunosuppressive agents, cyclosporine, corticosteroids, such as prednisone, chloroquine, hydroxychloroquine, sulfasalazine (sulphasalazopryine), gold salts, DMARDs, leflunomide, azathioprine, methotrexate, or D-penicillamine, TNFα blockers or antagonists, such as infliximab, or etanercept, or any biological agent targeting an inflammatory cytokine, such as anakinra, a molecule that blocks TNF receptors (e.g., pegsunercept), a molecule that blocks cytokine function(e.g., AMG719), a molecule that blocks LFA-1 function (e.g., efalizumab). Or nonsteroidal antiinflammatory drugs (NSAIDs), such as diflunisal, magnesium salicylate, salsalate, sodium salicylate, diclofenac, etodolac, fenoprofen, flurbiprofen, indomethacin, ketoprofen, ketorolac, meclofenamate, naproxen, nabumetone, phenylbutazone, piroxicam, sulindac, tolmetin, acetaminophen, ibuprofen, other COX inhibitors and specific Cox-2 inhibitors, meloxicam, or codeine phosphate, or acetyl salicylic acid, choline magnesium salicylate, or other analgesic agents such as propoxyphene napsylate, oxycodone hydrochloride, oxycodone bitartrate, tramadol, or dihydrofolic acid reductase inhibitor or other metabolic inhibitor.

The kit comprises a container with a label and/or instructions. Suitable containers include, for example, bottles, vials, and test tubes. The containers can be formed from a variety of materials such as glass or plastic. The container can have a sterile access port (for example the container can be an intravenous solution bag or a vial having a stopper pierceable by a needle such as a hypodermic injection needle). The container can hold a pharmaceutical composition of the invention.

The kit can also comprise a second container comprising one or more second agents as described herein and/or a pharmaceutically acceptable buffer, such as phosphate-buffered saline, Ringer's solution and dextrose solution. It may further include other materials desirable from a commercial and user standpoint, including other buffers, diluents, filters, needles, syringes, and package inserts with instructions for use.

The kit may also suitably include a label and/or instructions on, or associated with the container. The label can provide directions for carrying out the preparation of the binding proteins for example, dissolving of the dry powders, and/or treatment for a specific autoimmune disease or disease state.

The label and/or the instructions can indicate directions for either in vivo or in vitro use of the pharmaceutical composition. The label and/or the instructions can indicate that the pharmaceutical composition is used alone, or in combination with a second agent.

The label can indicate appropriate dosages for the binding proteins of the invention. For example, the label can indicate that dosages for a binding protein that is effective for enhancing the clearance of dead or dying cells is about 0.1 to 100 mg/kg weight of the subject.

The label and/or instructions can also indicate dosages for a second agent is about 1 to about 5000 mg/day.

The label and/or the instructions can also indicate that the pharmaceutical composition can be used alone, or in combination, with a second agent to reduce the symptoms of immune system diseases, autoimmune diseases, immunoproliferative diseases, graft-related disorders, graft versus host disease (GVHD) (e.g., such as may result from bone marrow transplantation, or stem cell engraftment, or solid organ transplantation, or as a sequellae of therapeutic attempts to induce immune tolerance), immune disorders associated with graft transplantation rejection, immune disorders associated with chronic rejection, immune disorders associated with tissue or cell allo- or xenografts (e.g., kidneys, skin, islets, muscles, hepatocytes, neurons, solid organs and the like), psoriasis, T cell lymphoma, T cell acute lymphoblastic leukemia, testicular angiocentric T cell lymphoma, benign lymphocytic angiitis, other forms of vasculitis, or autoimmune diseases such as lupus (e.g., lupus erythematosus, or cutaneous lupus), Hashimoto's thyroiditis, primary myxedema, Graves' disease, pernicious anemia, autoimmune atrophic gastritis, Addison's disease, diabetes (e.g. insulin dependent diabetes mellitus, type I diabetes mellitus, type II diabetes mellitus), Goodpasture's syndrome, glomerulonephritis, myasthenia gravis, pemphigus, Crohn's disease, sympathetic ophthalmia, autoimmune uveitis, multiple sclerosis, autoimmune hemolytic anemia, idiopathic thrombocytopenia, primary biliary cirrhosis, chronic action hepatitis, complications of hepatitis B or hepatitis C, ulcerative colitis, Sjogren's syndrome, inflammatory arthritis (e.g., rheumatoid arthritis, psoriatic arthritis or Reiter's syndrome), polymyositis, scleroderma, mixed connective tissue disease, and the like.

In a specific embodiment of the invention, the kit comprises a pharmaceutical composition comprising a pharmaceutically acceptable carrier and an effective amount of a first agent, wherein the first binding protein is an antibody of the invention.

The following examples are presented to illustrate the present invention, and to assist one of ordinary skill in making and using the same. The examples are not intended in any way to otherwise limit the scope of the invention.

### EXAMPLE 1

### Development of In Vivo Autoimmune Disease Model Systems to Test Protective Properties of an T15 IgM Antibodies

To test the effect of T15 IgM anti-PC Ab infusions on autoimmune pathogenesis, the lupus mouse model was used (NZWxBXSB)F1 (WXF1), which represent the progeny of male BXSB mice bred to female NZW mice purchased from a commercial source (Jackson Labs), which develops anti-phospholipid syndrome, autoimmune thrombocytopenia, and accelerated mortality from glomerulonephritis. These mice also develop classic IgG anti-nuclear antibodies against anti-native (ds) DNA, and anti-cardiolipin antibodies, and are also prone to accelerated atherosclerosis. For these investigations, the B cell hybridomas for the IgM antibodies were grown under serum-free conditions in hollow fiber bioreactors, and highly purified, endotoxin-low or -free antibodies were produced and then concentrated, under contract with a commercial vendor (National Cell Culture Center, Minneapolis, Minn.).

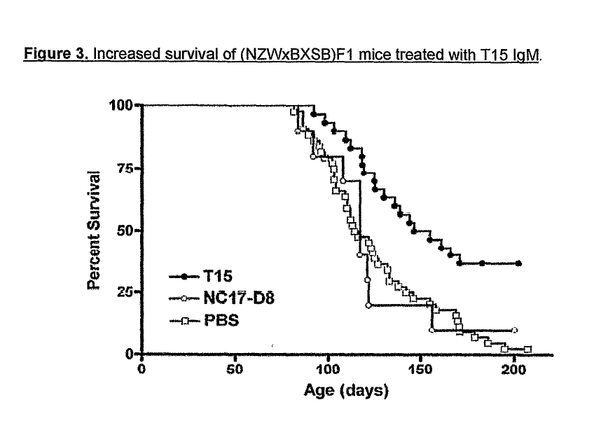

Increased Survival of (NZWxBXSB)F1 Mice Treated with T15 IgM. (FIG. 3).

To test whether T15 IgM treatments can affect the course of an autoimmuune disease, from 8 wks of life, different WXF1 mouse groups received treatments intraperitoneal (i.p.) every 2 wks, with 1.5 mg of T15 IgM (N=30) or phosphate buffered saline (PBS) (N=44), or 1.5 mg of an IgM isotype control of unrelated specificity (NC17-D8)(N=10) in saline. Kaplan-Meier plots of treated mice are shown. These treatment studies of lupus mice document statistically significant improved survival following treatment with the T15 IgM, compared to PBS treatment (p=0.0003 by logrank); and with T15 IgM treatment compared to NC17-D8 treatment (p<0.01 by logrank). There was no statistical difference in survival between lupus-prone WXF1 mouse groups that received the saline or the IgM isotype control (NC17-D8) treated groups.

These results, which showed that T15 IgM treatment significantly improved survival of WXF1 mice, present a definitive means for demonstrating the impact of a therapeutic intervention on autoimmune inflammatory pathogenesis that results in premature death.

While this initial dose treatment regimen was based on in vivo pharmacokinetic studies in C57BL/6 mice, the circulating half-life (i.e., t^{1/2}) of the T15 IgM was determined to be much shorter in WXF1 mice, as there was no detectable T15 IgM by ˜7 days after a dosing. These findings suggest that a more frequent dosing regimen, by providing a more consistent level of the therapeutic antibody should provide enhanced clinical benefits. Enhanced benefits should also result from the use of a form of an anti-PC antibody with a longer in vivo half-life, such as an IgG anti-PC antibody.

### EXAMPLE 2

### T15 IgM Enhances Complement Deposition on Dying Cells and Enhanced Apoptotic Clearance

To best test and quantitatively measure the properties of antibodies for their potential in vivo protective properties, a robust and relevant experimental system was developed to assess how a specific monoclonal IgM may affect the molecular and cellular mechanisms responsible for apoptotic clearance. muMT−/− mice are genetically manipulated mice that were previously made with homozygotic knockouts of an exon in the constant region of the immunoglobulin (Ig) mu heavy chain gene⁵⁵. This targeted mutation blocks B cell development at a B-cell precursor stage, so muMT−/− mice have no mature B cells and no circulating Ig, although other facets of their immune systems are not directly affected (47). Therefore, as there are no endogenous circulating immunoglobulins (Igs) to affect assays, a defined Ig sample can be introduced into the muMT−/− mouse and relevant biologic properties directly assessed.

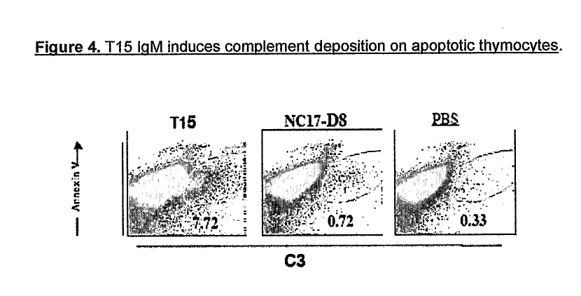

### FIG. 4. T15 μM Induces In Vitro Complement Deposition on Apoptotic Thymocytes.

To assess whether specific interactions with T15 IgM, but not IgM with unrelated binding specificities, the following test was performed. Apoptotic thymocytes (after etoposide induction), were incubated at 37° C. with 20% Ig-deficient (muMT) plasma, with an IgM at 20 μg/ml or PBS. After 15 min in culture, cells were washed and studied by flow cytometry. Only T15 IgM treatment lead to staining of apoptotic cells with the detection reagent, fluorochrome labeled goat anti-mouse C3, which identifies deposition of an early complement factor. No staining was detected if incubations with T15 IgM were instead performed in the presence of complement-deficient plasma or with media without Ca++. No complement deposition was detected if incubations instead used an IgM isotype control of irrelevant binding specificity. Together these findings documented the activity of the T15 IgM anti-PC antibody to mediate complement activation and cell deposition, which is known to require complement-containing plasma and sufficient local concentrations of calcium (Ca++).

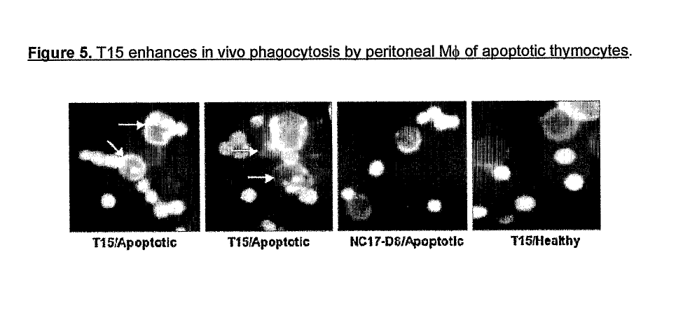

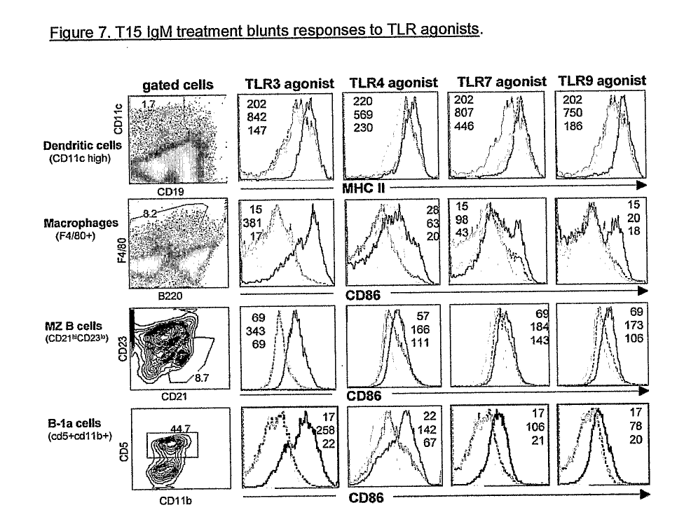

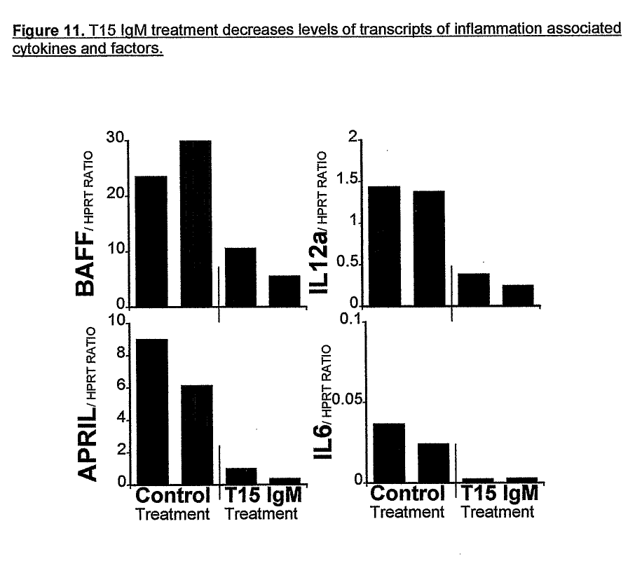

T15 IgM, like other anti-PC antibodies, specifically recognizes dead or dying cells (and apoptotic bodies)⁶. These studies clearly show that interactions of T15 IgM with apoptotic/necrotic cells results in specific complement deposition on these dead or dying cells (see FIG. 4), and this complementization is a form of opsonization that correlates with enhanced in vivo and in vitro clearance of dead and dying cells, as shown in (FIGS. 5&6), and with the capacity to blunt TLR mediated cell activation (see FIG. 7) and blunt the level of pro-inflammatory factors (see FIG. 11).

### FIG. 5. T15 μM Enhances In Vitro Phagocytosis of Apoptotic Thymocytes by Peritoneal

To assess the influence of T15 IgM on the clearance of dead and dying cells, muMT−/− mice were treated with saline or 1 mg of IgM ˜16 hr before in vivo instillation of labeled thymocytes, and later sacrificed (FIG. 5). Here, thymocytes were first labeled with the SNARF-1 fluoro-chrome (red). After harvest of the peritoneal cells, we performed cytospins and stained with the F4/80 FITC (green) detection reagent, which identifies macrophages, an important type of phagocytic cell. Mice received either apoptotic cells (generated by culture with etoposide), or healthy cells, which otherwise were immunologically compatible syngeneic cells. For cells recovered from the peritoneal cavities of treated muMT−/− mice, prior treatment with T15 IgM resulted in the clustering and formation of chains of apoptotic thymocytes, and greatly enhanced engulfment of these dead or dying cells by the phagocytic Mφ (left panels). These findings did not occur in mice that received the apoptotic cells and infusion of the IgM control of irrelevant binding specificity (NC17-D8 IgM). These findings were also not found in mice that received T15 IgM and then given healthy (freshly isolated) thymocytes (at right). These studies also demonstrated that after incubation with the T15 IgM, the apoptotic cells (but not freshly isolated thymocytes) were agglutinated into clusters/chains and became associated with Mφ. In these panels, arrows point to phagocytosed apoptotic thymocytes within Mφ. Hence, only the T15 IgM anti-PC antibody was shown to enhance the uptake and phagocytosis of dead or dying cells by the Mφ.

For these studies, (Ig-deficient) muMT−/− mice were used to assess the activities of T15 IgM anti-PC antibody, which binds dead or dying cells in vitro, compared with the NC17-D8 IgM that does not bind dead or dying cells. Taken together, these studies showed that when thymocytes undergoing apoptotic death were incubated with (complement factor containing) plasma from (Ig-deficient) muMT−/− mice, the addition of T15 IgM induced the deposition of complement on the apoptotic cells, as shown by staining with Annexin V that identifies dead or dying cells with characteristic membrane changes. In fact the relatively healthy (Annexin V-nonreactive) thymocytes were not coated with complement. Cells undergoing dexamethasone-induced apoptosis yielded the same findings of T15 IgM mediated complement deposition.

The therapeutic benefits from T15 IgM treatment therefore correlate with the in vitro capacity to enhance the deposition of early complement components onto dead and dying cells, which enhances their recognition, interaction and uptake by phagocytic cells, such as macrophages and dendritic cells. Signalling induced by complement factors deposited on these dead and dying cells, and their breakdown products, such as apoptotic blebs, could also alter the nature of the level of induced activation, and the capacity to provide cognate signals, and/or alter the types of cytokines released or induced by such macrophages or dendritic cells. These assays therefore provide a means to assess for properties of an antibody or recombinant protein that may predict for therapeutic benefits if infused at sufficient levels.

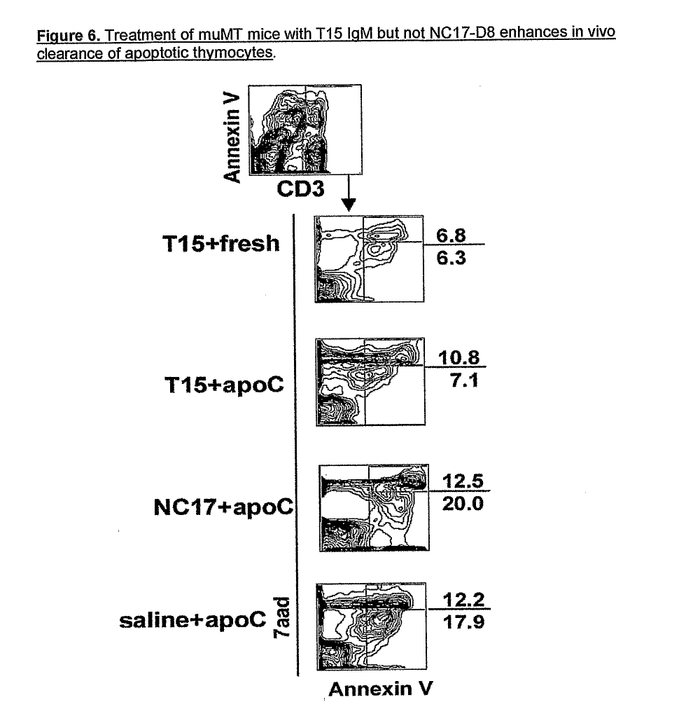

### FIG. 6. T15 IgM Enhances In Vivo Phagocytosis of Apoptotic Thymocytes by Peritoneal Mφ.

To assess whether T15 IgM also affects the in vivo clearance of dead and dying cells, an experimental system was used in which four days before the final assays, muMT−/− mice received intraperitoneal (i.p.) thioglycollate treatment to activate peritoneal Mφ. Mice then received 1 mg of a monoclonal IgM or saline intravenous (i.v.) 16 hr before i.p. instillation of fresh (i.e., healthy) or etoposide-induced apoptotic thymocytes, which were from immunologically compatible mice. At 10 minutes after i.p. instillation of these thymocytes, all peritoneal cells were recovered by lavage with ice cold HBSS/EDTA. Flow cytometric analyses were performed that used anti-CD3-fluorochrome staining to identify the transferred thymocytes, and other T cells. Cells at early stages of apoptosis were identified based on 7AAD non-staining and AnnexinV-reactivity (FIG. 6). Mice treated with T15 IgM were found to have greatly increased clearance (i.e., absent from the peritoneal cavity) of these dying/dead cells, compared to saline treatment (P=0.002), or compared to mice treated with control IgM of irrelevant binding specificity (NC17-D8) (P=0.004). T15 IgM treatment also enhanced the clearance of cells at later stages of death (i.e., 7AAD-reactive Annexin V-reactive) which may include necrotic cells. These studies therefore together show that T15 IgM treatment enhances the binding, uptake and engulfment by phagocytic cells, such as peritoneal Mφ.

### FIG. 7. T15 IgM Treatments Downregulate TLR Signaling in Mφ, DC and Some B Lymphocytes.

Studies were also performed to determine whether a limited duration (i.e., 2 weeks) of T15 IgM treatment can affect cellular responses to challenge with purified agonists for distinct Toll-like receptors (TLR), which trigger inflammatory responses and which have been implicated in the pathogenesis of inflammatory diseases and also inflammatory autoimmune diseases such as systemic lupus erythmatosus (SLE). For these studies, groups of adult C57BL/6 mice received three i.p. infusions (on days 0, 7 & 14) of 1.5 mg of T15 IgM or IgM isotype control of irrelevant specificity. At 12 hr before sacrifice on day 17, different groups of mice received saline or challenge with TLR3 agonist (Poly I:C, 100 ug), or TLR4 agonist (endotoxin, 100 ug) or TLR7 agonist (SM-360320, 300 ug)⁵⁶, or TLR9 agonist (phosphorothioate CpG oligo 1018, 200 ug⁵⁷). As shown in the panels in. FIG. 7, mean fluorescence indices (MFI) are indicated: top, control gray shaded area; mid, isotype control IgM followed by TLR agonist challenge, thick solid line; bottom, T15 treatment followed by same TLR agonist, red dashed line. Here, MFI is a numerical flow cytometric measure of the level of expression of these markers of cell activation, which can be induced by TLR triggering.

These results show that treatment with T15 IgM for two weeks blunts the cellular responses to challenge with purified ligands for distinct Toll-like receptors (TLR), as shown by a blunting (i.e., lowering) of the level of expression of cell-membrane-associated activation markers induced by in vivo ligand challenge. These TLR agonists, which are believed to be analogous to specific products from microbial pathogens and some self-ligands, are known to act as important triggers of both cells of the innate and adaptive immune system to amplify immune responses and induce inflammation. Hence, as shown in FIG. 7, these results provide direct evidence that by increasing the in vivo levels of an IgM anti-PC antibody one can greatly inhibit the capacity of dendritic cells (DCs), macrophages (Mφ), and certain types of mature B cells to become activated via specific cellular receptors. Among these cell types, only Mφ are important quantitative contributors to apoptotic cell clearance. However, all of these cell types express TLR and complement receptors. Hence, treatment with a T15 IgM may provide benefits by altering interactions involving complement deposition onto apoptotic cell associated components, and determinants, with cells that express complement receptors and can be triggered to contribute to inflammatory responses or which can dampen inflammatory responses.

As shown in FIG. 7, prior treatment with T15 IgM, but not isotype control, was shown to blunt the capacity for subsequent induction of membrane-associated activation markers by the TLR agonist challenge, with examples shown for the expression of the co-stimulatory molecules, CD86 and MHC class II. To limit the opportunity for secondary induced changes, a minimal interval was used between in vivo TLR agonist challenge and immediate ex vivo analysis (i.e., 12 hr). While T15 IgM anti-PC antibody treatment induced the functional blockade of macrophages (Mφ) and dendritic cells (DCs) in their responses to each of the different TLR agonists, in these pilot studies T15 induced inhibition was more complete for the TLR3 agonist, TLR4 agonist and the TLR9 agonist than for the TLR 7 ligand. For these studies SM-360320 was used for the TLR7 agonist, which has 100-fold greater activity than resiquimod⁵⁶. In addition, for F4/80+ cells, a marker for macrophages (Mφ), contours indicate subsets differ in their TLR responsiveness and/or degree of protection with T15 IgM treatment. Therefore, not all Mφ and DC subsets may be equally affected. Therefore it is possible that T15 IgM treatment might blunt responses to some TLR agonists, and for some sets of DC and Mφ without affecting others. Hence, T15 IgM may affect pathogenesis and blunt inflammatory responses and/or pathologic autoimmune responses, while generalized immunosuppression may not occur. In addition to decreasing responsiveness to these TLR agonists, this same T15 IgM regimen also increased representation of splenic CD 11c+ DC and pilot studies indicated that this treatment affected representation of both splenic CD8α+ and plasmacytoid DC although other types of DC are also affected.

In these studies the responses within the B cell compartment was also assessed. While splenic marginal zone (MZ) B cells had significant TLR ligand responses, these responses were also blunted by T15 IgM treatment, while follicular B cells (B220⁺CD23^{hi}CD21^{lo}) were not as greatly affected. In the peritoneal cavity, the responses of B-1a cells to in vivo challenge with TLR agonists were also blunted by T15 IgM treatment, compared to responses in mice that instead received control treatment. In general, Mφ and DC were more affected by treatment with anti-PC antibody than were the lymphocytes in these studies.

Cumulatively, these studies show that a monoclonal IgM anti-PC antibody that can selectively recognize dead or dying cells in vivo, enhances the binding, uptake and engulfment of these cells by phagocytic cells, such as peritoneal Mφ. These treatments also blunt the capacity of phagocytic and non-phagocytic cells to be triggered by factors that otherwise induce inflammation. These studies also suggest that complement plays central roles in these activities, as different types of mononuclear cells with the highest levels of complement receptors (e.g., macrophages, dendritic cells, marginal zone B cells and B-1a cells) were also the most affected by in vivo treatments.

These results show that application of this invention can yield global immuno-modulatory effects, which have the potential to block pathways implicated in the pathogenesis of inflammatory diseases and autoimmune diseases.

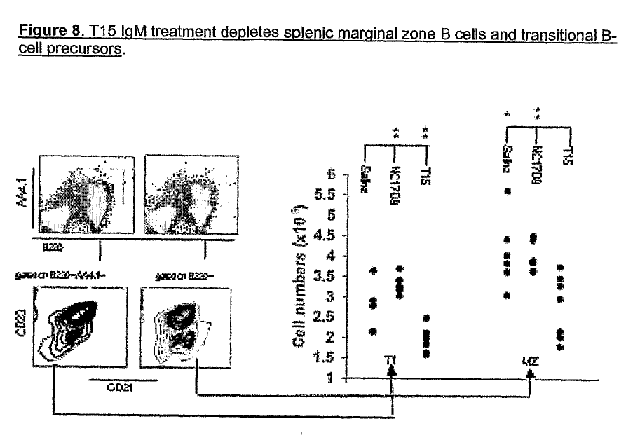

### FIG. 8. T15 IgM Treatment Depletes Splenic Marginal Zone B Cells and Transitional B-Cell Precursors.

To assess the effect of T15 IgM treatment on the cellular representation within the B-cell compartment of the adaptive immune system, data were compiled from two independent experiments in which separate groups of adult C57BL/6 mice received 1.5 mg of T15 IgM, or an IgM of irrelevant specificity (NC17-D8) or saline, given on days 0, 7 and 14 and sacrificed on day 17. In each group, there were a total of mice 6-7 mice. From surveys of mononuclear cells in the spleen, two weeks of T15 IgM treatment was shown to induce a significant decrease in the representation of splenic marginal zone (MZ) B cells, defined based on the surface phenotype of B220+CD21^{hi}CD23^{lo} by flow cytometry (p<0.015, two-tailed t test). There was also a significant depletion of early transitional B-cell precursors in the spleen (i.e., T1) defined based on B220+AA4.1^{hi} CD24^{hi}CD21^{lo} (p<0.007, two-tailed t test)^{58;59} (FIG. 8).

Mature follicular B cells were less affected by T15 IgM infusions. In many respects these T15 IgM induced changes resemble those reported to be induced with polyclonal IgM into secreted IgM-deficient mice⁶⁰, although such a mechanism was not discussed in this report.

While similar observations were made from the studies with T15 IgM, a different mechanism is suggested as contributing to the altered representation of mature B cells within the spleen, as T15 IgM treatment decreased the representation of MZ B cells in a strain of mice, C57BL/6 mice that otherwise have normal B cells, which is unlike the earlier reported study⁶⁰. Because MZ B cells are reported to at times contribute to pathologic autoimmune diseases, these experimental findings provide additional evidence that monoclonal anti-PC antibody treatments may provide therapeutic benefits, by interfering with the recruitment of these MZ B lymphocytes into disease-associated responses.

Based on this evidence of decreased representation of B-cell precursors in the spleen after T15 IgM treatment, it was hypothesized these treatments may affect this peripheral lymphoid compartment by decreasing the repletion of these peripheral B cells from the B-cell precursors within the bone marrow (BM), the central compartment.

This hypothesis was confirmed by examination of the central compartments in these same mice, as flow cytometric analyses demonstrated that T15 IgM treatment also induced the significant depletion of pre-B/immature B cells (B220^{lo/+} sIgM^{lo/+} sIgD^{lo/-}) compared to saline or IgM isotype control treated groups (N=6/group, p<0.006, two-tailed t test). In contrast, these T15 IgM treatments did not affect the levels of recirculating follicular (IgM+IgD^{hi}) B cells in the bone marrow, which are present at this site because they have recirculated from the periphery.

To further characterize the mechanism responsible for the loss of B-lineage cells, DNA labeling (i.e., bromodeoxyuridine, BrDu) studies were performed that determine the rate of cell generation/turnover. T15 IgM treated C57BL/6 mice had significantly lower levels of BrDu labeling in splenic B cells, compared to saline or isotype control treatment. These studies provided evidence that T15 IgM treatment can induce lower levels of repletion in MZ B cells and T1 cell compartments in the spleen, and also in B-cell precursors in the bone marrow, likely due to the deficient generation or survival of these B-lineage subpopulations, presumably due to lower levels of B-cell specific survival factors, like BAFF, as shown in FIG. 11.

### EXAMPLE 3

### IgG Antibodies from the pIGG Vector Display Bind PC with High Affinity (FIG. 9) and Recognize Dead and Dying Cells by Flow Cytometry (FIG. 10)

To assess the binding properties of recombinant T15 IgG antibodies, produced using the pIGG expression vector that contains cloned T15 variable regions and cloned antibody constant regions, a solid phase immunoassay was first performed. Herein, ELISA wells were coated with PC conjugated to bovine serum albumin (PC-BSA), that were then blocked and Ig samples of interest added in replicate and using serial dilutions. As all recombinant IgG contained mouse kappa light chains, the levels of binding in these assays were determined by subsequent development with an anti-mouse (ms) κ light chain immunoglobulin (Ig) specific detection reagent. The positive IgG control was PCG1-14, which is a mouse IgG antibody from a cell line generated by immunization with PC-KLH. For the negative control, the MOPC21 is an IgG of irrelevant binding specificity that does not recognize PC. All other antibodies shown in FIG. 9 are recombinant IgG proteins expressing the T15 V regions with different H constant regions, that were produced using the pIGG vector. The T15-human (hu) IgG1 is a human chimeric antibody that expresses the murine T15 V regions and human IgG1 constant region. By this assay, all recombinant T15 based IgG antibodies were shown to express equivalent PC binding activity. In specific, based on the binding curve, the recombinant IgG antibodies that express the variable regions of the T15 parental clone (e.g., T15-IgG2a and T15 IgG2a D265A) were shown to have high binding activity for the recognition and binding of the PC-protein conjugate, PC-BSA (FIG. 9). These activities are also comparable to those previously described for other anti-PC antibodies that also recognize and bind to apoptotic cells but not healthy cells⁶.

The activity of the recombinant T15-IgG2a, generated from the pIGG vector, was also assessed in flow cytometry assays, which showed that this T15 IgG antibody recognizes a determinant on dead and dying thymocytes. As shown in FIG. 10, in the top panels the T15 IgG anti-PC antibody specificity is shown for total thymocytes undergoing etoposide-induced apoptosis. In the bottom panels, the T15 IgG2a antibody also recognizes early apoptotic cells (i.e., Annexin V-pos 7AAD-neg), which is inhibited by PC (not shown). Reactivity was demonstrated by using a tagged IgG gamma heavy chain-specific reagent. (FIG. 10). This assay, which requires relatively high binding reactivity to demonstrate binding, also showed that the T15 IgG recognized and bound cells at early stages of apoptotic death (i.e., 7AAD-non reactive Annexin V reactive). The demonstrated recognition by T15 IgG antibodies of cells at early stages of apoptotic death is especially important as it would be most desirable to facilitate clearance of such cells and breakdown products at early stages of death, to prevent cellular progression to late stages and/or necrosis that might instead support an inflammatory host response.

### EXAMPLE 4

### T15 IgM Treatment Decreases Levels of RNA Transcripts of Inflammation Associated Cytokines and Factors

To determine whether two weeks of T15 IgM treatment can affect cytokine levels, quantitative transcript assays using the Taqman system were used. The same two week IgM or control treatment regimen, used in the studies illustrated in the flow cytometry studies in FIG. 7, were used to treat C57BL/6 mice that were sacrificed on d17. These mice did not receive TLR agonist challenge. Cytokine levels in whole spleen of two adult C57BL/6 mice in each group were determined by Taqman with commercial primer sets (PE Applied Biosystems), and depicted after normalization for HPRT transcript levels (FIG. 11). While the levels of specific transcripts differ somewhat in each individual mouse, T15 IgM treatment resulted in reductions at baseline (i.e., related to the homeostatic set point) in BAFF transcripts and APRIL transcripts, which are key B-cell survival factors. These pilot studies also provided evidence that T15 IgM reduced baseline pro-inflammatory IL-6 IL-12α, which are pro-inflammatory cytokines (FIG. 11). As shown, T15 IgM treatment was shown to affect the baseline levels of expression of a range of cytokines in the spleen. These studies showed that in vivo T15 IgM treatments downregulates homeostatic levels of pro-inflammatory and B-cell prosurvival cytokine transcripts in the spleen (FIG. 11).

Pilot studies have also shown that in adult C57BL/6 mice receiving control treatments and a challenge with poly IC (TLR3 agonist) or CpG (TLR9) induce increased splenic IL-6 and IL-12α transcript levels when assayed at 12 hr after in vivo challenge, while such responses were blunted in mice that received the two week treatment regimen with T15 IgM (not shown). Taken together, these studies indicate that T15 IgM treatments can downregulate baseline levels of some cytokines implicated in inflammatory diseases, and also blunt the capacity for TLR agonists to trigger ro-inflammatory cytokine responses.

The capacity for treatment with an anti-PC antibody to lower baseline levels of pro-inflammatory cytokine and lower levels of BAFF and APRIL may also contribute to the decrease in the representation of certain B-lineage cells that followed after two weeks of T15 IgM treatment, as shown in FIG. 8. These decreased cytokine transcript levels may reflect the direct or indirect effects of the T15 IgM anti-PC antibody in vivo treatment on macrophages and/or dendritic cells that can be the sources of some or all of these factors.

### REFERENCES

1. Kuenkele, S., Beyer, T. D., Voll, R. E., Kalden, J. R., and Herrmann, M. Impaired clearance of apoptotic cells in systemic lupus erythematosus: challenge of T and B cell tolerance. Curr Rheumatol Rep 5(3), 175-7. 2003.
2. Cline, A. M. and Radic, M. Z. Apoptosis, subcellular particles, and autoimmunity. Clin Immunol 112(2), 175-82. 2004.
3. Fadok, V. A. and Henson, P. M. Apoptosis: giving phosphatidylserine recognition an assist—with a twist. Curr Biol 13(16), R655-7. 2003.
4. Fadok, V. A., Bratton, D. L., and Henson, P. M. Phagocyte receptors for apoptotic cells: recognition, uptake, and consequences. J Clin Invest 108(7), 957-62. 2001.
5. Martin, S., J., Reutelingsperger, C. P., McGahon, A. J., Rader, J. A., van Schie, R. C., LaFace, D. M., and Green, D. R. Early redistribution of plasma membrane phosphatidylserine is a general feature of apoptosis regardless of the initiating stimulus: inhibition by overexpression of Bcl-2 and Abl. J Exp Med 182(5), 1545-56. 1995.
6. Shaw, P. X., C. S. Goodyear, M.-K. Chang, J. Witztum, and G. Silverman. The Autoreactivity of Anti-Phosphorylcholine Antibodies for Atherosclerosis-Associated Neo-antigens and Apoptotic Cells. J. Immunol. 170:6151-7. 2003.
7. Dumitriu, I. E., Baruah, P., Manfredi, A. A., Bianchi, M. E., and Rovere-Querini, P. HMGB1: guiding immunity from within. Trends Immunol 26(7), 381-7. 2005.
8. Kim, S., Elkon, K. B., and Ma, X. Transcriptional suppression of interleukin-12 gene expression following phagocytosis of apoptotic cells. Immunity 21(5), 643-53. 2004.
9. Mevorach, D., Mascarenhas, J. O., Gershov, D., and Elkon, K. B. Complement-dependent clearance of apoptotic cells by human macrophages. J Exp Med 188(12), 2313-20. 1998.
10. Korb, L. C. and Ahearn, J. M. C1q binds directly and specifically to surface blebs of apoptotic human keratinocytes: complement deficiency and systemic lupus erythematosus revisited. J Immunol 158(10), 4525-8. 1997.
11. Quartier, P., Potter, P. K., Ehrenstein, M. R., Walport, M. J., and Botto, M. Predominant role of IgM-dependent activation of the classical pathway in the clearance of dying cells by murine bone marrow-derived macrophages in vitro. Eur J Immunol 35(1), 252-60. 2005.
12. Boes, M., Schmidt, T., Linkemann, K., Beaudette, B. C., Marshak-Rothstein, A., and Chen, J. Accelerated development of IgG autoantibodies and autoimmune disease in the absence of secreted IgM. Proc Natl Acad Sci USA 97(3), 1184-9. 2000.
13. Ehrenstein, M. R., Cook, H. T., and Neuberger, M. S. Deficiency in serum immunoglobulin (Ig)M predisposes to development of IgG autoantibodies. J Exp Med 191(7), 1253-8. 2000.
14. Kim, S. J., Gershov, D., Ma, X., Brot, N., and Elkon, K. B. I-PLA(2) activation during apoptosis promotes the exposure of membrane lysophosphatidylcholine leading to binding by natural immunoglobulin M antibodies and complement activation. J Exp Med 196(5), 655-65. 2002.
15. Lutz, H. U., Stammler, P., and Fasler, S. Preferential formation of C3b-IgG complexes in vitro and in vivo from nascent C3b and naturally occurring anti-band 3 antibodies. J Biol Chem 268(23), 17418-26. 1993.
16. Hamett, W. and Hamett M. M. Phosphorylcholine: friend or foe of the immune system. Immunology Today 20(3), 125-129. 1999.
17. Feeney, A. J. Lack of N regions in fetal and neonatal mouse immunoglobulin V-D-J junctional sequences. J Exp Med 172(5), 1377-90. 1990.
18. Sigal, N. H., Pickard, A. R., Metcalf, E. S., Gearhart, P. J., and Klinman, N. R. Expression of phosphorylcholine-specific B cells during murine development. J. Exp. Med. 146, 933-948.1977.
19. Claflin, J. L. and Cubberley, M. Clonal nature of the immune response to phosphocholine. VI. Molecular uniformity of a single idiotype among BALB/c mice. J. Immunol. 121, 1410-1415. 1978.
20. Claflin, J. L. and Rudikoff, S. Uniformity in a clonal repertoire: a case for a germ-line basis of antibody diversity. Cold Spring Harb Symp Quant Biol 41 Pt 2, 725-34. 1977.
21. Quintans, J. and Cosenza, H. Antibody response to phosphorylcholine in vitro. II. Analysis of T-dependent and T-independent responses. Eur J Immunol 6(6), 399-405. 1976.
22. Briles, D. E., Forman, C., Hudak, S., and Claflin, J. L. Anti-phosphorylcholine antibodies of the T15 idiotype are optimally protective against Streptococcus pneumoniae. J. Exp. Med. 156, 1177-1185. 1982.
23. McDaniel, L. S., Benjamin, W. H. J., Forman, C., and Briles, D. E. Blood clearance by anti-phosphocholine antibodies as a mechanism of protection in experimental pneumococcal bacteremia. J. Immunol. 133, 3308-3312. 1984.
24. Shaw, P. X., Hörkkö, S., Chang, M. K., Curtiss, L. K., Palinski, W., Silverman, G. J., and Witztum, J. L. Natural antibodies with the T15 idiotype may act in atherosclerosis, apoptotic clearance, and protective immunity. J Clin Invest 105(12), 1731-40. 2000.
25. Chang, M. K., Bergmark, C., Laurila, A., Hörkkö, S., Han, K. H., Friedman, P., Dennis, E. A., and Witztum, J. L. Monoclonal antibodies against oxidized low-density lipoprotein bind to apoptotic cells and inhibit their phagocytosis by elicited macrophages: evidence that oxidation-specific epitopes mediate macrophage recognition. Proc Natl Acad Sci USA 96(11), 6353-8. 1999.
26. Fadok, V. A., Bratton, D. L., Frasch, S. C., Warner, M. L., and Henson, P. M. The role of phosphatidylserine in recognition of apoptotic cells by phagocytes. Cell Death Differ 5(7), 551-62. 1998.
27. Kagan, V. E., Borisenko, G. G., Tyurina, Y. Y., Tyurin, V. A., Jiang, J., Potapovich, A. I., Kini, V., Amoscato, A. A., and Fujii, Y. Oxidative lipidomics of apoptosis: redox catalytic interactions of cytochrome c with cardiolipin and phosphatidylserine. Free Radic Biol Med 37(12), 1963-1985. 2004.
28. Nicolo, D., Goldman, B. I., and Monestier, M. Reduction of atherosclerosis in low-density lipoprotein receptor-deficient mice by passive administration of antiphospholipid antibody. Arthritis Rheum 48(10), 2974-8. 2003.
29. Freireich, E. J., Gehan, E. A., Rall, D. P., Schmidt, L. H., and Skipper, H. E. Quantitative comparison of toxicity of anticancer agents in mouse, rat, hamster, dog, monkey, and man. Cancer Chemother Rep 50(4), 219-44. 1966.
30. Binder, C., S. Horkko, A. Dewan, M.-K. Chang, E. Kieu, C. Goodyear, P. Shaw, W. Palinski, J. Witztum, and G. Silverman. Pneumococcal vaccination decreases atherosclerotic lesion formation: Molecular mimicry between oxidized LDL and Streptococcus pneumoniae. Nature Medicine 9:736-43. 2003.
31. Rader, C., Popkov, M., Neves, J. A., and Barbas, C. F. 3rd. Integrin alpha(v) beta3 targeted therapy for Kaposi's sarcoma with an in vitro evolved antibody. FASEB J 16(14), 2000-2. 2002.
32. Kearney, J. F., Barletta, R., Quan, Z. S., and Quintans, J. Monoclonal vs. heterogeneous anti-H-8 antibodies in the analysis of the anti-phosphorylcholine response in BALB/c mice. Eur. J. Immunol. 11, 877-883. 1981.
33. Clynes, R. and Ravetch, J. V. Cytotoxic antibodies trigger inflammation through Fc receptors. Immunity 3(1), 21-6. 1995.
34. Harlow, E. and Lane, D. Using Antibodies. A laboratory manual. 99. Cold Spring Harbor, N.Y., Cold Spring Harbor Laboratories Press. 1999.
35. Neuberger, M. S., Williams, G. T., and Fox, R. O. Recombinant antibodies possessing novel effector functions. Nature 312(5995), 604-8. 1984.
36. Sharon, J., Gefter, M. L., Manser, T., Morrison, S. L., Oi, V. T., and Ptashne, M. Expression of a VHC kappa chimaeric protein in mouse myeloma cells. Nature 309(5966), 364-7. 1984.
37. Tan, L. K., Oi, V. T., and Morrison, S. L. A human-mouse chimeric immunoglobulin gene with a human variable region is expressed in mouse myeloma cells. J Immunol 135(5), 3564-7. 1985.
38. Fell, H. P., Yamold, S., Hellstrom, I., Hellstrom, K. E., and Folger, K. R. Homologous recombination in hybridoma cells: heavy chain chimeric antibody produced by gene targeting. Proc Natl Acad Sci USA 86(21), 8507-11. 1989.
39. Jones, P. T., Dear, P. H., Foote, J., Neuberger, M. S., and Winter, G. Replacing the complementarity-determining regions in a human antibody with those from a mouse. Nature 321(6069), 522-5. 1986.
40. Riechmann, L., Clark, M., Waldmann, H., and Winter, G. Reshaping human antibodies for therapy. Nature 332(6162), 323-7. 1988.
41. Verhoeyen, M., Milstein, C., and Winter, G. Reshaping human antibodies: grafting an antilysozyme activity. Science 239(4847), 1534-6. 1988.
42. Carter, P., Presta, L., Gorman, C. M., Ridgway, J. B., Henner, D., Wong, W. L., Rowland, A. M., Kofts, C., Carver, M. E., and Shepard, H. M. Humanization of an anti-p185HER2 antibody for human cancer therapy. Proc Natl Acad Sci USA 89(10), 4285-9.1992.
43. Sims, M. J., Hassal, D. G., Brett, S., Rowan, W., Lockyer, M. J., Angel, A., Lewis, A. P., Hale, G., Waldmann, H., and Crowe, J. S. A humanized CD18 antibody can block function without cell destruction. J Immunol 151(4), 2296-308. 1993.
44. Caron, P. C., Laird, W., Co, M. S., Avdalovic, N. M., Queen, C., and Scheinberg, D. A. Engineered humanized dimeric forms of IgG are more effective antibodies. J Exp Med 176(4), 1191-5.1992.
45. Shopes, B. A genetically engineered human IgG mutant with enhanced cytolytic activity. J Immunol 148(9), 2918-22. 1992.
46. Wolff, E. A., Schreiber, G. J., Cosand, W. L., and Raff, H. V. Monoclonal antibody homodimers: enhanced antitumor activity in nude mice. Cancer Res 53(11), 2560-5. 1993.
47. Vaughan, T. J., Osbourn, J. K., and Tempest, P. R. Human antibodies by design. Nat Biotechnol 16(6), 535-9. 1998.
48. Winter, G., Griffiths, A. D., Hawkins, R. E., and Hoogenboom, H. R. Making antibodies by phage display technology. Annu Rev Immunol 12, 433-55. 1994.
49. Burton, D. R. and Barbas, C. F. 3rd. Human antibodies from combinatorial libraries. Adv Immunol 57, 191-280. 1994.
50. Lonberg, N. Human antibodies from transgenic animals. Nat Biotechnol 23(9), 1117-25. 2005.
51. Amon, et. al. Monoclonal antibodies for immunotargeting of drugs in cancer therapy. In Monoclonal antibodies and cancer therapy. R. Reisfeld and et al., editors. Alan R. Liss, Inc., NY. 243-265. 1985.
52. Hellstrom, et. al. Antibodies for drug delivery. In Controlled drug delivery. Marcel Dekker, hic., 623-53. 1987.
53. Thorpe, et. al. Antibody carriers of cytotoxic agents in cancer therapy. A review. In Monoclonal antibodies '84: Biological and clinical applications. 475-506. 1984.
54. Thorpe, P. E. and Ross, W. C. The preparation and cytotoxic properties of antibody-toxin conjugates. Immunol Rev 62, 119-58. 1982.
55. Kitamura, D., Roes, J., Kuhn, R., and Rajewsky, K. A B cell-deficient mouse by targeted disruption of the membrane exon of the immunoglobulin mu chain gene. Nature 350(6317), 423-6. 1991.
56. Kurimoto, A., Ogino, T., Ichii, S., Isobe, Y., To be, M., Ogita, H., Takaku, H., Sajiki, H., Hirota, K., and Kawakami, H. Synthesis and evaluation of 2-substituted 8-hydroxyadenines as potent interferon inducers with improved oral bioavailabilities. Bioorg Med Chem 12(5), 1091-9. 2004.
57. Sato, Y., Roman, M., Tighe, H., Lee, D., Corr, M., Nguyen, M. D., Silverman, G. J., Lotz, M., Carson, D. A., and Raz, E. Immunostimulatory DNA sequences necessary for effective intradermal gene immunization. Science 273, 352-354. 1996.
58. Rolink, A. G., Tschopp, J., Schneider, P., and Melchers, F. BAFF is a survival and maturation factor for mouse B cells. Eur J Immunol 32(7), 2004-10. 2002.
59. Loder, F., Mutschler, B., Ray, R. J., Paige, C. J., Sideras, P., Torres, R., Lamers, M. C., and Carsetti, R. B cell development in the spleen takes place in discrete steps and is determined by the quality of B cell receptor-derived signals. J Exp Med 190(1), 75-89.1999.
60. Baker, N. and Ehrenstein, M. R. Cutting edge: selection of B lymphocyte subsets is regulated by natural IgM. J hmmunol 169(12), 6686-90. 2002.

1. A method for inhibiting a pathologic autoimmune response in a subject comprising contacting dead or dying cells exposing an antigen selected from the group consisting of an antigen having a phosphorylcholine (PC) determinant, an antigen having a phosphatidyl serine (PS) determinant, an antigen having a MDA determinant, and an antigen having a cardiolipin in the subject, with a binding protein that recognizes and binds the antigen that is exposed on the dead or dying cells, thereby inhibiting the pathologic autoimmune response in the subject.

2. The method of claim 1, wherein the pathologic autoimmune response is an inflammatory response.

3. The method of claim 1, wherein the binding protein is an antibody or fragment or variant thereof.

4. (canceled)

5. (canceled)

6. The method of claim 3, wherein the antibody is a T15 antibody which comprises a native or modified constant region.

7.-9. (canceled)

10. The method of claim 1, further comprising multiple binding proteins that recognize and bind multiple antigens that are exposed on the dead or dying cells, so that the dead or dying cells are cleared from the subject thereby inhibiting the pathologic autoimmune response in the subject.

11. A method for treating an immune system disease in a subject by inhibiting a pathologic autoimmune response in a subject by the method of claim 1 or 10, using the binding protein in an amount effective to enhance clearance of dead or dying cells from the subject and/or inhibit an inflammatory response in the subject.

12. (canceled)

13. (canceled)

14. The method of claim 11, wherein the immune system disease is selected from a group consisting of psoriasis, Hashimoto's thyroiditis, primary myxedema, Graves' disease, pernicious anemia, autoimmune atrophic gastritis, Addison's disease, diabetes, Goodpasture's Syndrome, myaesthenia gravis, pemphigus, Crohn's disease, autoimmune uveitis, multiple sclerosis, autoimmune hemolytic anemia, idiopathic thrombocytopenia, primary biliary cirrhosis, chronic active hepatitis, ulceratis colitis, Sjogren's syndrome, arthritis, polymyositis, scleroderma, an inflammatory disease or disorder, lupus erythematosus, lupus nephritis and glomerulonephritis.

15. (canceled)

16. (canceled)

17. The method of claim 11, wherein the immune system disease is a complication of therapeutic transplantation.

18. The method of claim 17, wherein the complication of therapeutic transplantation is graft versus host disease (GVHD).

19. The method of claim 17, wherein the complication of therapeutic transplantation disease is transplant rejection.

20. The method of claim 19, wherein the transplant rejection is allograft transplant rejection.

21. The method of claim 14, wherein the immune system disease is macular degeneration.

22. A method for reducing levels of circulating autoantibodies in a subject by the method of claim 1.

23. A method for ameliorating an autoimmune disease associated with impaired clearance of dead or dying cells, or associated with production of proinflammatory soluble or cell associated factors by inhibiting a pathological autoimmune response in the subject by the method of claim 1.

24. A method for ameliorating a disease associated with production of proinflammatory soluble or cell associated factors by inhibiting a pathological autoimmune response in the subject by the method of claim 1.

25. A recombinant protein comprising the antigen-binding region of a T15 antibody.

26. The recombinant protein of claim 25, wherein the protein is a human/murine chimeric antibody.

27. The recombinant protein of claim 25, wherein the antigen-binding region is joined to a native or modified IgG tail.

28. An Fab, F(ab′)₂, or Fv fragment of the recombinant protein of claim 25.

29. A bispecific antibody with a binding specificity for two different antigens, one of the antigens being that with which the recombinant protein of claim 25 binds.

30. The recombinant protein of claim 25, wherein the protein is conjugated to a therapeutic agent to form a recombinant protein conjugate.

31. The recombinant protein of claim 30, wherein the therapeutic agent is an anti-tumor drug, a toxin, a radioactive agent, a second antibody or an enzyme.

32. A combination of an immunoconjugate comprising the recombinant protein of claim 25 linked to an enzyme capable of converting a prodrug into a cytotoxic drug, and said prodrug.

33. A composition comprising a combination of an immunoconjugate comprising the recombinant protein of claim 25 linked to an enzyme capable of converting a prodrug into a cytotoxic drug, and said prodrug.

34. A method of alleviating symptoms associated with chemotherapy comprising administering to a patient a pharmaceutically effective amount of the recombinant protein of claim 25 so as to inhibit inflammation and thereby alleviate signs and symptoms, complications associated with chemotherapy.

35.-60. (canceled)
